(19) United States
(12) Patent Application Publication
(10) Pub. No.: US 2022/0161257 A1
(43) Pub. Date: May 26, 2022

(54) INTEGRATION OF WORLD-TO-CHIP INTERFACES WITH DIGITAL MICROFLUIDIC FOR BACTERIAL TRANSFORMATION AND ENZYMATIC ASSAYS

(71) Applicant: VALORBEC, SOCIÉTÉ EN COMMANDITE, MONTRÉAL (CA)

(72) Inventors: Ehsan MOAZAMI, Montréal (CA); James PERRY, Montréal (CA); Guy SOFFER, Montréal (CA); Steve SHIH, Montréal (CA)

(73) Assignee: VALORBEC, SOCIÉTÉ EN COMMANDITE, MONTRÉAL, QC (CA)

(21) Appl. No.: 17/600,084

(22) PCT Filed: Mar. 27, 2020
(86) PCT No.: PCT/IB2020/052958

§ 371 Date: Sep. 29, 2021

Related U.S. Application Data
(60) Provisional application No. 62/826,387, filed on Mar. 29, 2019.

(51) Int. Cl. B01L 3/00; B01L 7/00

(52) U.S. Cl. CPC B01L 3/502715; B01L 7/52; B01L 3/502707; B01L 3/502784; B01L 2300/0645; B01L 2200/0673; B01L 2200/16; B01L 2300/0867; B01L 2400/0478; B01L 2300/1822; B01L 2300/0663; B01L 2300/165

(57) ABSTRACT

Systems, devices and methods for integrating world-to-chip interfaces with digital microfluidics for bacterial transformation and enzymatic assays are described herein. The devices include a microfluidic device having a first plate comprising a cell culture region for maintaining a cell culture and a reservoir for storing reagents to induce at least a portion of the cell culture or to mix with other reagents and a second plate spaced apart from the first plate, the second plate defining one or more openings extending through the second plate. The device may include a reagent well coupled to the second plate. The reagent well may be configured to refill the reservoir on the first plate with liquid reagent via the one or more openings of the second plate. The device may also include a thermoelectric module (TEM) coupled to the first plate or the second plate for managing temperature control of the device.

### CROSS-REFERENCE TO RELATED APPLICATIONS

The present application claims priority to U.S. application No. 62/826,387 filed on Mar. 29, 2019. This application is hereby incorporated by reference in its entirety.

### TECHNICAL FIELD

The embodiments disclosed herein relate to microfluidic systems, devices and methods, and more specifically, to systems, devices and methods for integrating world-to-chip interfaces with digital microfluidics for bacterial transformation and enzymatic assays.

### BACKGROUND

Microfluidics is a valued liquid handling tool for biological and chemical assays. The advantages of low reagent consumption and automation is particularly useful for point-of-care diagnostics, cell-based assays for drug discovery, and immuno-based enzymatic assays.¹⁻³ Recently, digital microfluidics (DMF) have emerged as a platform to manipulate droplets without the need for pumps, moving parts, or valves. Droplets on DMF devices are individually addressable using an electrostatic force which allow for a number of fluidic operations such as splitting, merging and mixing to be performed on the device—a great advantage over other microfluidics paradigms. Given these functionalities, DMF platforms have developed to automate magnetic-bead based immunoassays,⁴ cell-based assays,⁵⁻⁶ as well as tackle the work-flows of synthetic biology including DNA assembly and transformation.⁷⁻⁸ Despite the attractive capabilities of DMF, a re-occurring issue lies in ‘world-to-chip’ interfacing. Typically, ‘world-to-chip’ interfacing accounts for fluidic interconnects—i.e. to deliver fluids from the macro- to the micro-device. ‘World-to-chip’ to be any external component that interfaces with the device that will enable facile automated operations on the device and minimize manual intervention. Hence, samples between device and user are, as well as provide the appropriate thermal conditions for a given biological reaction on a DMF device.

Temperature control is critical for many biological protocols namely, polymerase chain reaction (PCR) and protein crystallization⁹⁻¹¹ as well as heat-shock transformation¹². The vast majority of studies requiring temperature control use hot-plates, water baths or thermal cyclers. These systems facilitate both homogeneous temperature regulation and linear temperature profiles—often with a high degree of accuracy. However, since these systems are not designed with the specifications of DMF in-mind they pose a limited degree of effectiveness when used with DMF. There are studies which report strategies to incorporate thermal electric coolers (TEC) for synthetic biology applications,⁸ but these studies do not incorporate closed-loop control. In fact, closed-loop control requires gain-tuning processes to achieve steady-state output. This tuning process can be difficult for users not familiar with control theory and therefore it is a persistent roadblock preventing wide adoption of this technique. To the best of our knowledge, a closed-loop system for controlling TEC with digital microfluidics have not been developed.

The second limitation for DMF (and possibly for all microfluidic systems) is the development of fluidic interfaces for delivering liquids. It is notoriously difficult to continuously deliver reagents to the reservoirs for continuous and repetitive dispensing, especially for long-term device usage and to store a large volume (>10 mL). In previous work, off-chip pressure sources are used to dispense liquids directly to a DMF device reservoir.^{12,13} Another option is to pre-load dried reagents and re-hydrate them as they are needed instead of repetitive dispensing.¹⁴ Currently, a popular technique to improve “world-to-chip” interfaces is the use of fluidic connector components fabricated using 3D printers. 3D printing provides a convenient one-step manufacturing approach for fabricating microfluidic devices¹⁵⁻¹⁶ and greatly expands the options available to solve “world-to-chip” interfacing. In fact, for microchannel-based techniques, 3D printed fluidic port connectors (or valves) store and deliver reagents to the fluidic channels.¹⁷⁻¹⁸ Despite wide use of 3D printed connector components in microfluidics, its use has been limited in DMF.

In response to “world-to-chip” interface challenges associated with digital microfluidics, a new strategy to implement a 3D printed fluidic port for reagent delivery has been developed which enables consistent replenishment of reagent reservoirs. In addition, a TEC based, closed-loop temperature control system for a DMF platform has been developed, providing the necessary thermal requirements for a variety of biological assays. Both of these systems serve to improve the “world-to-chip” interface tool-box for DMF. To demonstrate these principles, a bacterial transformation protocol on DMF was performed. Furthermore, the utility of the systems described here with an enzymatic assay are shown to show how the reagent delivery systems disclosed herein may improve the reproducibility of droplet dispensing on device while performing an enzymatic assay on-chip.

### SUMMARY

Systems, devices and methods for integrating world-to-chip interfaces with digital microfluidics for bacterial transformation and enzymatic assays are described herein

According to one aspect, a microfluidic device is described herein. The microfluidic device includes:

a first plate comprising:
a cell culture region for maintaining a cell culture; and
a reservoir for storing a liquid reagent to induce at least a portion of the cell culture or to mix with other reagents;

a second plate spaced apart from the first plate, the second plate defining one or more openings extending through the second plate; and

a reagent well coupled to the second plate, the reagent well configured to refill the reservoir on the first plate with the liquid reagent via the one or more openings of the second plate.

According to another aspect, a microfluidic system is described herein. The microfluidic system includes:

a microfluidic device comprising:
a first plate comprising:
   a cell culture region for maintaining a cell culture; and
   a reservoir for storing reagents to induce at least a portion of the cell culture or to mix with other reagents; and
a second plate spaced apart from the first plate, the second plate defining one or more openings extending through the second plate; and

a thermoelectric module (TEM) coupled to the first plate or the second plate, the TEM for controlling a temperature of the device.

According to another aspect, a method of controlling a temperature of a region of a microfluidic device is described herein. The method includes:

comparing a set point temperature to a measured temperature, the measured temperature measured by a temperature sensor;

determining a temperature set point error based on the measured temperature and the set point temperature;

providing the temperature set point error to a proportional-integral-derivative (PID) controller; and

calculating, by the PIC controller, an output value based on the temperature set point error and a set of parameters for proportional, integral and derivative temperature control.

These and other features and advantages of the present application will become apparent from the following detailed description taken together with the accompanying drawings. However, it should be understood that the detailed description and the specific examples, while indicating preferred embodiments of the application, are given by way of illustration only, since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1A shows a perspective view of a digital microfluidics (DMF) system according to one embodiment, the system including a DMF device integrated with two world-to-chip interfaces: a reagent delivery system and a thermoelectric module (TEM).

FIG. 1B shows a perspective view of the device of FIG. 1A and a side view of reagent droplets being dispensed from the reagent well of the device of FIG. 1A.

FIG. 2A shows a top view of the DMF device of FIG. 1A, according to another embodiment showing the alignment of the well with respect to the reservoirs.

FIG. 2B shows A series of images from a movie to show the operation of the 3D printed well.

FIG. 2C shows results showing droplet dispensing using with and without the reagent delivery system for three liquids with varying viscosities: water, methanol, and LB media (with Pluronics F-68 0.05%).

FIG. 3A shows a block diagram of the TEC showing the closed-loop temperature model, according to one embodiment that includes the PID control with a thermodynamically modelled plant design which include electrical, Peltier and thermal effects.

FIG. 3B shows simulation results showing optimal Kₚ, Kᵢ, and K_{d} constants for a target temperature of 42° C.

FIG. 4A shows an infrared image showing the heating (42° C.) and cooling regions (4° C.) on a DMF device used for bacterial transformation.

FIG. 4B shows a comparison between simulated and experimental results for open- and closed-loop control.

FIG. 4C shows graphs providing the transformation efficiency for single experiments.

FIG. 4D shows graphs providing the transformation efficiency for co-transformation experiments.

FIG. 5A is a schematic showing the chemical scheme of the enzymatic assay.

FIG. 5B shows data generated on-chip showing the effects of temperature on the enzymatic assay at two different temperatures (23° C. and 30° C.).

FIG. 5C shows experimental accuracy for ITO-DMF and ITO-well-DMF driven enzymatic assays; the enzymatic assay was evaluated for three substrate concentrations (5, 10, 20 μM) with the enzyme concentration remaining constant. All samples were analyzed in triplicates with error bars showing ±1 S.D.

FIG. 6A is a plasmid map of pFAB4876,

FIG. 6B is a plasmid map of pSB1C3,

FIG. 6C is a plasmid map of pET-EGL.

FIG. 7A shows a perspective view of the device of FIG. 1, according to another embodiment.

FIG. 7B shows a design of the 3D printed well, according to one embodiment.

FIG. 8A shows a reagent delivery interface for digital microfluidic devices, specifically a typical two-plate setup used for digital microfluidics to automate liquid handling on device.

FIG. 8B shows a ITO-well-DMF is the new world-to-chip system for reagent delivery on device consisting of a 3D printed well integrated on top of the ITO top-plate which is aligned to an electrode (i.e. reservoir) on the device.

FIG. 9A shows an impedance measurement circuit to measure fluid volume on the device. where the circuit consists of a resistor and the potential (V_{read}) is measured to correlate the amount of fluid to the potential.

FIG. 9B shows a calibration curve to correlate volume and voltage.

FIG. 10A shows a DMF design layout for bacterial transformation.

FIG. 10B shows a DMF design layout for enzymatic assay experiments.

FIG. 11A shows a graph showing the temperature output when the system is given a low rise time, high fluctuation, and low stability.

FIG. 11B shows a graph showing the temperature output when the system is given a low rise time, high overshoot, and no stability. All experiments had a target temperature of 42° C.

FIG. 12A shows a heat shock protocol for transforming chemically competent E. col, according to one embodiment.

FIG. 12B shows a graph of plotted temperature following a heat shock protocol of a 50 μL cell/DNA sample, according to one embodiment.

FIG. 12C shows a formula for the approximation of the transfer function for heating a body.

FIG. 13A shows a device setup modeled in COMSOL, according to one embodiment.

FIG. 13B shows a graph showing a comparison of simulated temperature result on the droplet and temperature sensor data.

FIG. 14A shows a graph of temperature profiles of PID controlled TEC/DMF with (τ=17) and without (τ=0.5) time constant adjustment with a set point of 42° C.

FIG. 14B shows a bar graph showing efficiencies resulting from time constant values compared with tube efficiency.

FIGS. 15A and 15B show the results of tests testing different time constants with a maximum set point of 37° C.

The skilled person in the art will understand that the drawings, further described below, are for illustration purposes only. The drawings are not intended to limit the scope of the applicant's teachings in any way. Also, it will be appreciated that for simplicity and clarity of illustration, elements shown in the figures have not necessarily been drawn to scale. For example, the dimensions of some of the elements may be exaggerated relative to other elements for clarity. Further aspects and features of the example embodiments described herein will appear from the following description taken together with the accompanying drawings.

### DETAILED DESCRIPTION

Various systems and methods are described below to provide an example of at least one embodiment of the claimed subject matter. No embodiment described below limits any claimed subject matter and any claimed subject matter may cover systems and methods that differ from those described below. The claimed subject matter are not limited to systems and methods having all of the features of any one system and method described below or to features common to multiple or all of the systems and methods described below. Subject matter that may be claimed may reside in any combination or sub-combination of the elements or process steps disclosed in any part of this document including its claims and figures. Accordingly, it will be appreciated by a person skilled in the art that a system or method disclosed in accordance with the teachings herein may embody any one or more of the features contained herein and that the features may be used in any particular combination or sub-combination that is physically feasible and realizable for its intended purpose.

Furthermore, it is possible that a system or method described below is not an embodiment of any claimed subject matter. Any subject matter that is disclosed in a system or method described herein that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

It will also be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

It should be noted that terms of degree such as “substantially”, “about” and “approximately” as used herein mean a reasonable amount of deviation of the modified term such that the result is not significantly changed. These terms of degree should be construed as including a deviation of the modified term, such as 1%, 2%, 5%, or 10%, for example, if this deviation would not negate the meaning of the term it modifies.

Furthermore, the recitation of any numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made, such as 1%, 2%, 5%, or 10%, for example, if the end result is not significantly changed.

It should be noted that the term “coupled” used herein indicates that two elements can be directly coupled to one another or coupled to one another through one or more intermediate elements.

It should also be noted that, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Systems, devices and methods for integrating world-to-chip interfaces with digital microfluidics for bacterial transformation and enzymatic assays.

Specifically, in some embodiments, microfluidic devices are described herein.

In some embodiments described herein, the reagent well is vertically spaced apart from the reservoir.

In some embodiments described herein, the reagent well is laterally spaced apart from the reservoir.

In some embodiments described herein, the reagent well is coupled to a top of the second plate.

In some embodiments described herein, the reagent well is coupled to a top of the second plate directly above the one or more openings of the second plate.

In some embodiments described herein, the first plate comprises an ancillary reservoir and the reagent well is configured to deliver the liquid reagent to the ancillary reservoir.

In some embodiments described herein, the reagent well is aligned with a center or an edge of the ancillary reservoir.

In some embodiments described herein, the reagent well comprises a plunger for dispensing droplets of the liquid reagent.

In some embodiments described herein, the plunger is a screw configured to fit within the reagent well and the liquid reagent is dispensed from the reagent well by applying an external pressure to the plunger.

In some embodiments described herein, the external pressure is a downward pressure.

In some embodiments described herein, the reagent well is 3D printed and the plunger is fitted within the reagent well.

In some embodiments, microfluidic systems including one or more microfluidic devices and a thermoelectric module (TEM) are described herein.

In some embodiments of these systems, the TEM is coupled to the second plate.

In some embodiments of these systems, the TEM is positioned underneath the second plate.

In some embodiments of these systems, the TEM is a Peltier module.

In some embodiments of these systems, the microfluidic device further comprises a temperature sensor to receive temperature information from the TEM.

In some embodiments of these systems, the TEM is controlled by a closed-loop temperature model.

In some embodiments of these systems, the system further comprises a PID controller to control the TEM.

In some embodiments of these systems, the system further comprises an aluminum heat block.

In some embodiments of these systems, the aluminum heat block is positioned between the TEM and the microfluidic device.

In some embodiments of these systems, the temperature sensor is positioned within the aluminium heat block.

In some embodiments, methods of controlling a temperature of a region of a microfluidic device are described herein

In some embodiments, the methods further include measuring the measured temperature with a temperature sensor positioned in a heat block before comparing the set point temperature to the measured temperature.

In some embodiments, the set point temperature is a value subtracted from the measured temperature from the temperature sensor.

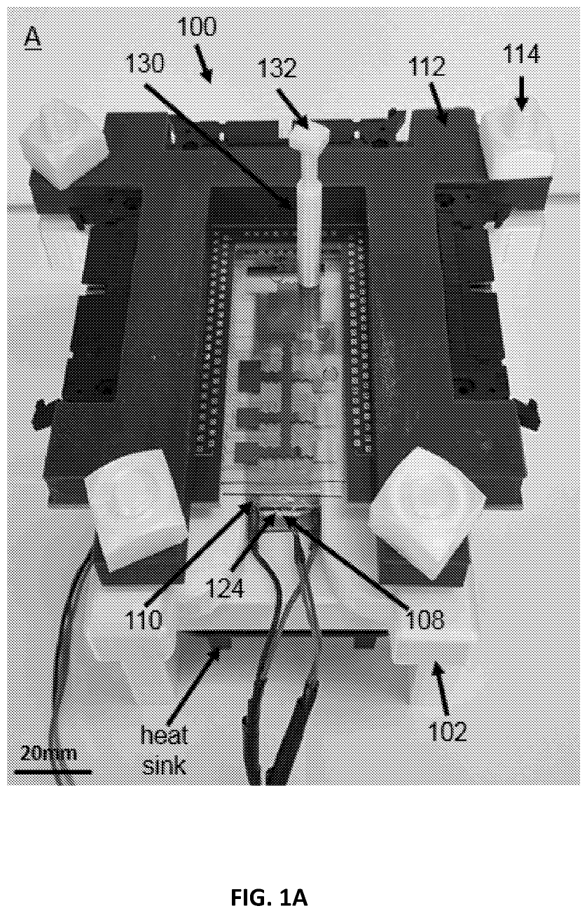

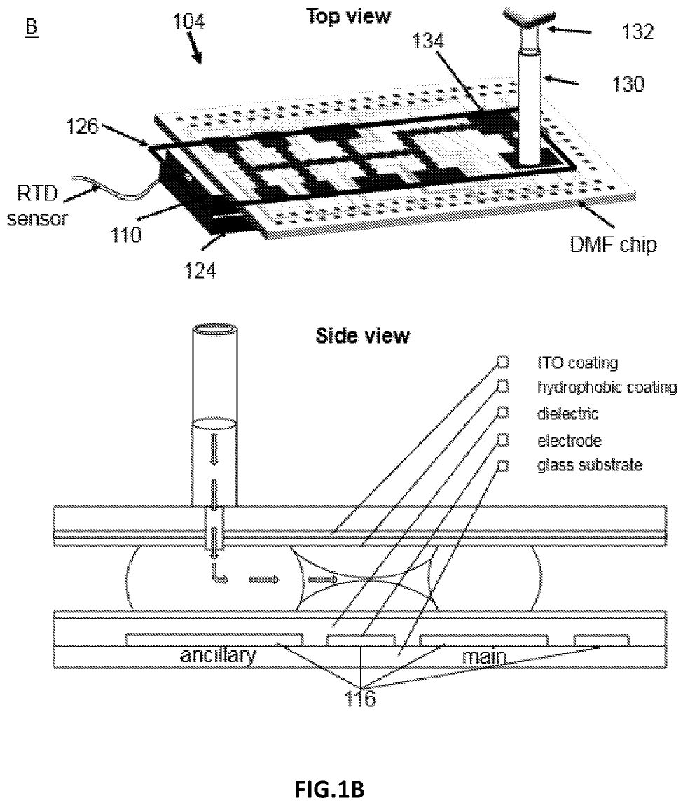

Referring now to FIG. 1, shown therein is a digital microfluidic system.

Herein, the integration of two world-to-chip interfaces for digital microfluidics are described. Namely, microfluidic systems and devices integrating reagent delivery and temperature control systems are described herein

Referring to FIG. 1A, a system 100 is shown therein. System 100 is generally formed by three-dimensional (3D) printing a bottom holder 102 used to interface a device (such as but not limited to a 50×75 mm device) 104, a thermoelectric module (e.g. cooler) 124, a temperature sensor 108, and an aluminum heat block 110. A top 3D printed cover 112 is mated to the bottom holder 102 with four fasteners 114 (e.g. fitting screws) and sandwiches a pogo pin holder and the device 104. The addition of the top cover 112 with fasteners 114 provides tight contact of the pogo pin board (not shown) to the device 104.

A top view of the device 104 is shown in FIG. 1B. As depicted, the digital microfluidic device 104 consists of electrodes 116 and each electrode 116 is wired to a contact pad (not shown). The array of contact pads on the side of the device 104 is connected to the pogo pin board (which is interfaced with an automation system; see refs^{5, 19-20} for a description). Device 104 is assembled with a top-plate (with or without well) and droplets are manipulated on the DMF device 104 by applying an AC field between the top and bottom plates.

Temperature control of the device 104 is managed by the thermoelectric module (TEM) (i.e. Peltier module) 124 which can be positioned underneath the device 104 with a temperature sensor 108 for closed-loop control.

Furthermore, a reagent well 130 is attached to the top plate to refill the reservoir 134 and to dispense reproducible droplets (see for example the side view shown in FIG. 1B).

Both of these world-to-chip interfaces, reagent well 130 and the TEM 124, may represent advances over the previous DMF device configurations which do not include reagent delivery to reservoirs with refilling of the reservoirs on the device 104 or a closed-loop control scheme for the TEM 124 (see, for example, Table 3 comparing current “world-to-chip” DMF work to work described herein).²²⁻²³

In current digital microfluidic systems, reagent delivery has been alleviated by the addition of reservoirs or loading pads on devices. Users may manually pipette the reagents directly onto the reservoir and use automated sequences to dispense liquids from the reservoir.^{5, 19, 24} As shown by several groups,²⁵⁻²⁶ dispensing from reservoirs is generally not a reproducible process (i.e. dispensing volumes have high variability).

Current solutions to alleviate the variability in the dispensing process is to integrate capacitative²⁶ or image-based feedback²⁰ control which can measure droplet volume via impedance or image-based algorithms respectively.

In some embodiments, the reservoir 134 can be refilled after every dispensing action. Refilling the reservoir 134 may reduce the variability in the dispensing process.

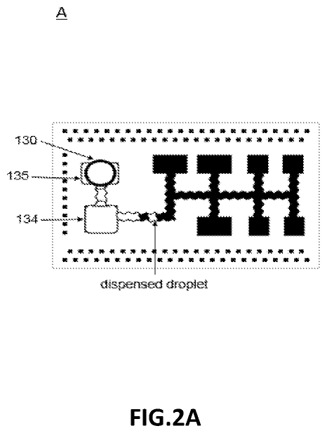

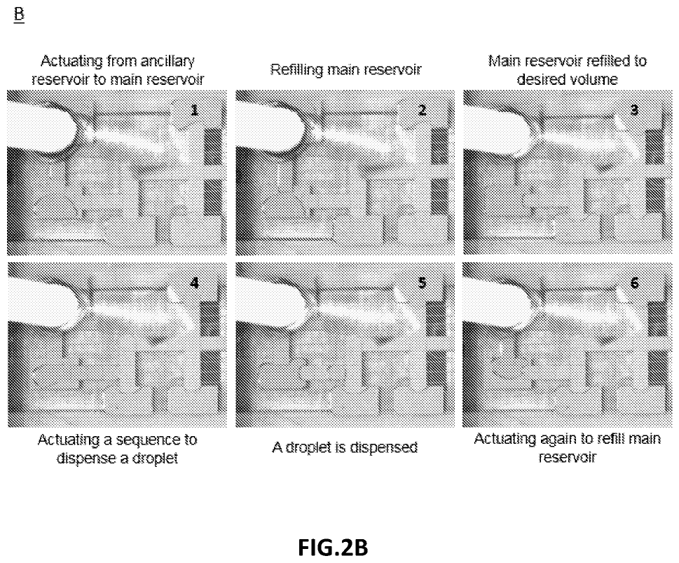

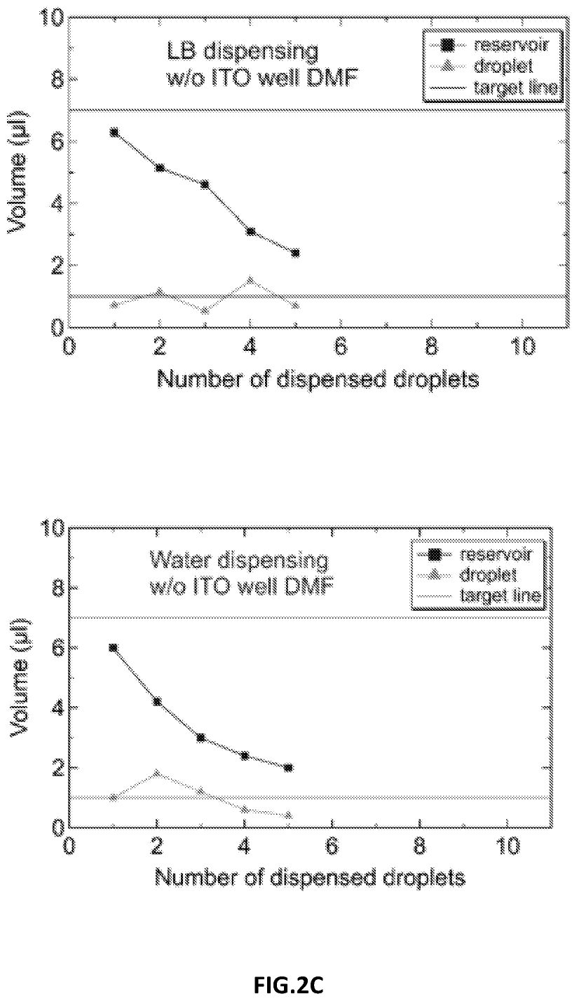

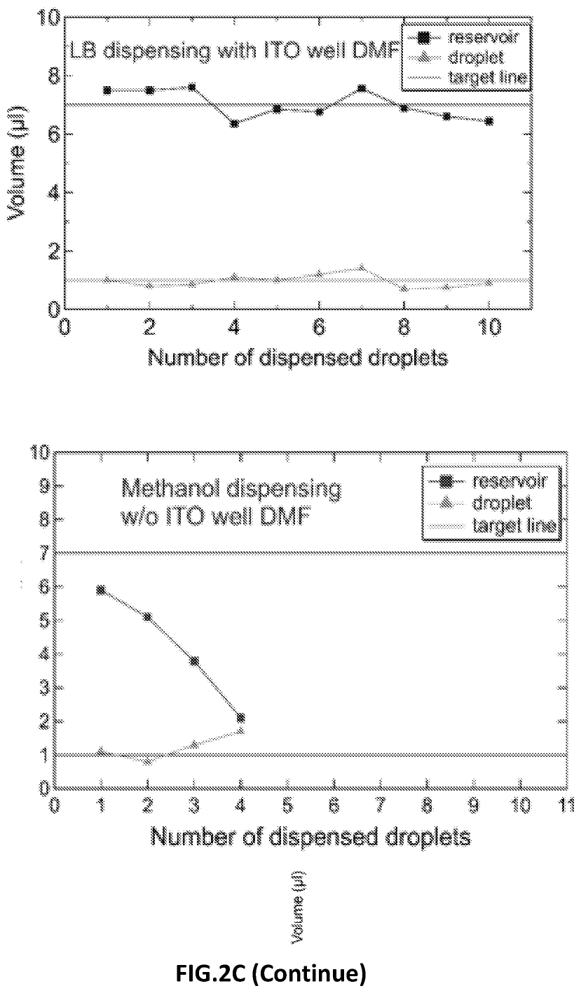

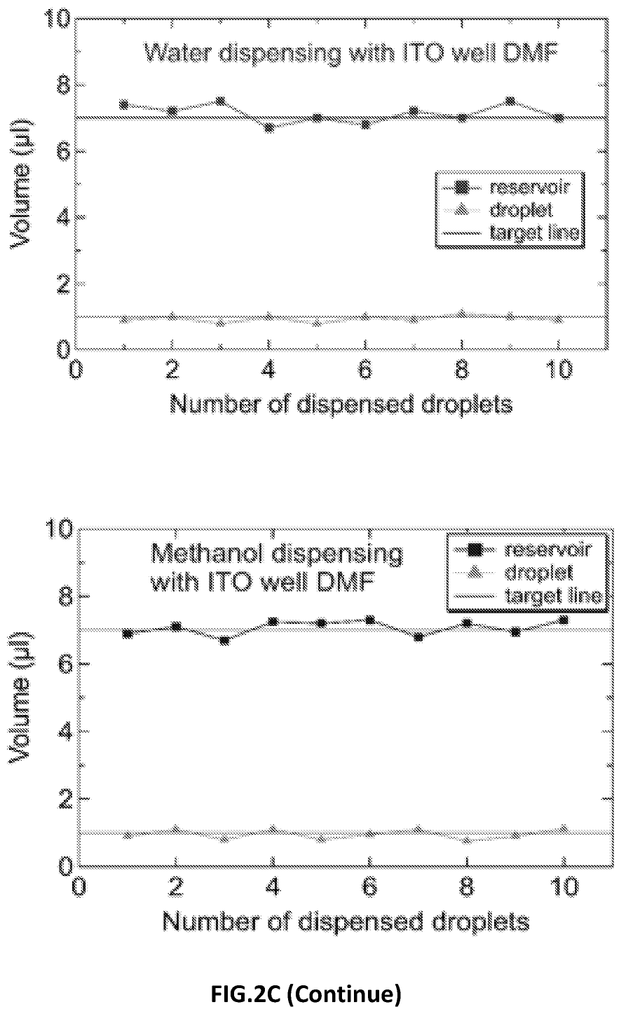

As shown in FIG. 2, shown therein is a reagent well 130 mated to a top of the ground plate 126 (e.g. indium tin oxide (ITO) ground plate). The ground plate 126 defines an aperture (e.g. a drilled-hole) that forms an inlet aligned to a reservoir 134 on-chip. Using a reagent well such as reagent well 130, devices such as device 104 can hold larger volumes (˜400 μL) of reagent than a typical reservoir (which hold ˜1-10 μL) on the chip.

In some embodiments, the reagent well 130 is cylindrical. In some embodiments, the reagent well 130 can be a 3D printed well.

In some embodiments, the device 104 including the reagent well 130 may be referred to as a ‘ITO-well-DMF’ and the liquid in the reagent well 130 may be used to refill the reservoir 134 on-chip, for instance by rotating a 3D printed screw (i.e. plunger) 132 further down the well.

The refilling process may be repeated when there has been reduction in liquid volume on the reservoir 134.

In some embodiments of the reagent well 130, the well 130 is capable of continuously delivering reagents to the reservoir 134 that will allow repetitive droplet dispensing. In some embodiments of the reagent well 130, the well 130 is capable of enabling reproducible droplet dispensing from the reservoir.

In some embodiments, the reagent well 130 is positioned to overlap the reservoir 134. In some embodiments, when the reagent well 130 directly overlaps the reservoir 134 (or partially overlaps), the fluid from the well 130 may not be confined to the reservoir 134 and will spread elsewhere on the device. In this configuration, the main reservoir 134 may be frequently overfilled (>7 μL) which can cause significant difficulties in dispensing unit droplets. A motorized component to automatically deliver the liquids may alleviate this,^{15, 28-29}.

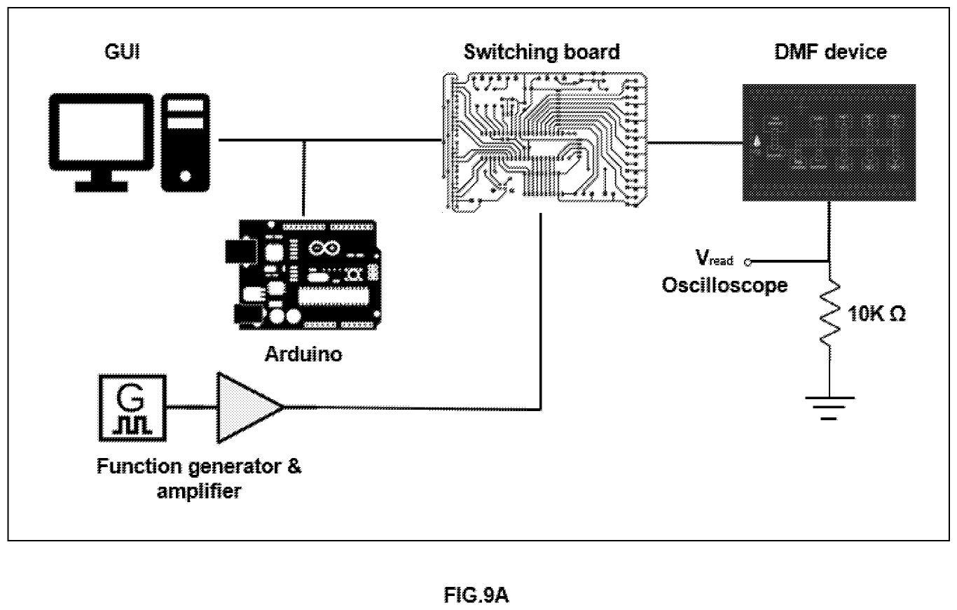

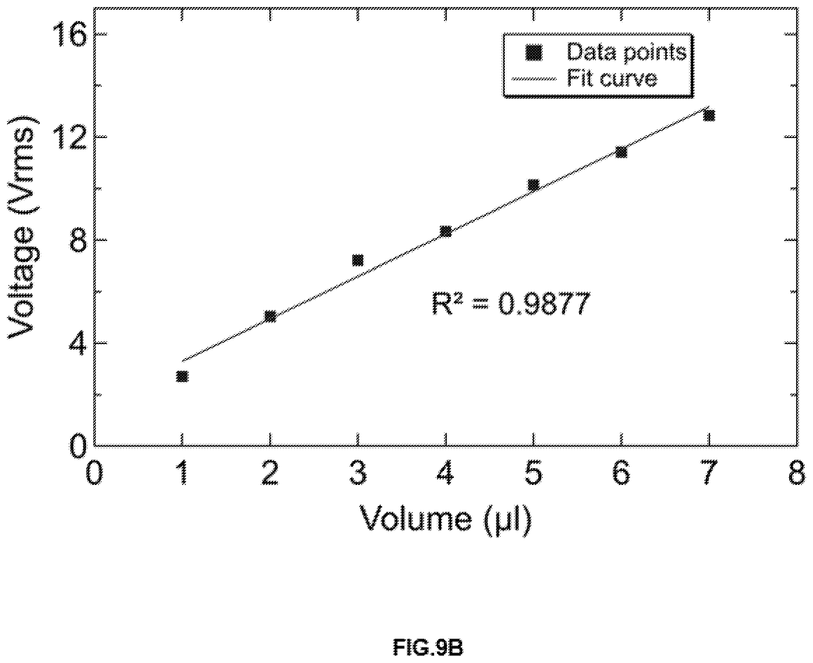

In the embodiments described herein, an ancillary (i.e. secondary) reservoir 135 is shown to refill the main reservoir 134 and an impedance circuit 136 is included to measure the volume (see FIG. 2A for device design and FIG. 9A for impedance circuit). Ancillary reservoir 135 may facilitate easy refilling without any external motors or pumps.

The process of reagent delivery follows generally four steps (as shown in FIG. 2B): (1) liquid from the 3D printed well 130 is delivered to the ancillary reservoir 135 (e.g. by rotating the screw 132), (2) liquid from the ancillary reservoir 135 is “pulled” out of the ancillary reservoir 135 and forms a liquid neck. The liquid neck merges with the liquid in the main reservoir 134, (3) when the volume in the main reservoir 134 reaches a target volume of 7 μL (i.e. V_{read}=12.9 V; FIG. 9b) in the main reservoir 134, the liquid neck is broken off by actuating the ancillary 135 and main reservoir 134. With only the main reservoir 134 (i.e. without an ancillary reservoir 135), a significant amount of trial-and-error may be required to reach the target reservoir volume. The excess time to refill the main reservoir 134 to the target volume typically fouls the surface of device 104 which prevents droplets to be actuated in that area. With the ancillary reservoir 135, a systematic process is introduced (i.e. without trial-and-error) which minimized the biofouling on the surface of the device and allowed further droplet manipulation.

After the refilling process described above, refilling reservoirs to improve the reproducibility of the dispensing process from the reservoir on-chip was tested. As shown in FIG. 2c, are six graphs showing the utility of the reagent well 134 and the precision of dispensing using the device 104 and ITO-DMF configurations for three types of liquids ranging in viscosity (e.g. LB media, water, and methanol). In the ITO-DMF configuration, droplets can be repeatedly dispensed from the main reservoir 134 until it was not possible to dispense any more droplets.

In these examples, the maximum number of droplets dispensed with an initial reservoir volume of 7 μL was five droplets with a target volume of ˜1 μL. In the device 104, the main reservoir 134 can be refilled after a droplet is dispensed from the reservoir 134. Two results are shown in FIG. 2c: (1) the device 104 (i.e. refilling the main reservoir) provides continuous dispensing—10 droplets may be dispensed (compared to only 5 droplets without the well) to show the capabilities of repetitive dispensing, but it can be potentially used to dispense more droplets. (2) The precision of the dispensed droplets in the device 104 (RSD=11.62%, 16.78%, 15.32% for LB, water, methanol respectively) was higher than the precision of devices lacking a reagent well 130 (RSD=19.61%, 49.29%, 35.41% for LB, water, methanol respectively) which demonstrates the reproducibility of using the reagent well.

There are a number of factors that suggest that there is a fundamental change occurring on the device 104 (e.g., dielectric degradation, contact angle saturation, etc. . . . ) that can change the dispensed droplet volume.²⁵ These are inevitable changes on the device and integrating a reagent well 130 (to our knowledge) to refill reservoirs and to ensure reproducible droplet dispensing on-chip.

A second world-to-chip interface is included in device 104 to control temperature on chip. There are numerous papers that have discussed the integration of micro-heaters (i.e. TEC elements) to establish a uniform or gradient temperatures in a given region on a microfluidic device.^{5, 30-32} However, in current microfluidic studies, the parameters used for the TEC are optimized for their own biological or chemical application. There has not been a fully modeled and characterized TEC control system that allows for a quick analysis, design, and optimization. In fact, a significant amount of trial-and-error is required to optimize the control-loop parameters and design. A model-based approach can reduce the time and the number of iterations to tune the control-loop parameters to meet the demand specifications on-chip.

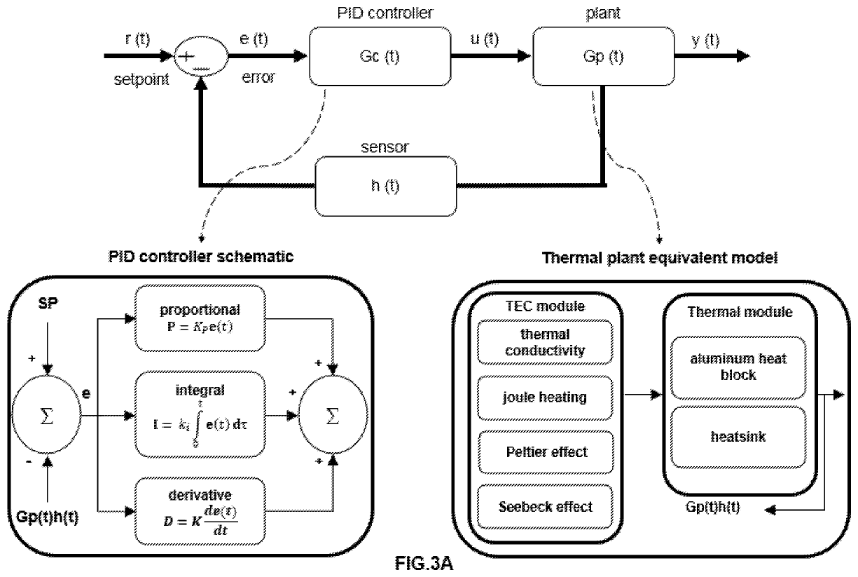

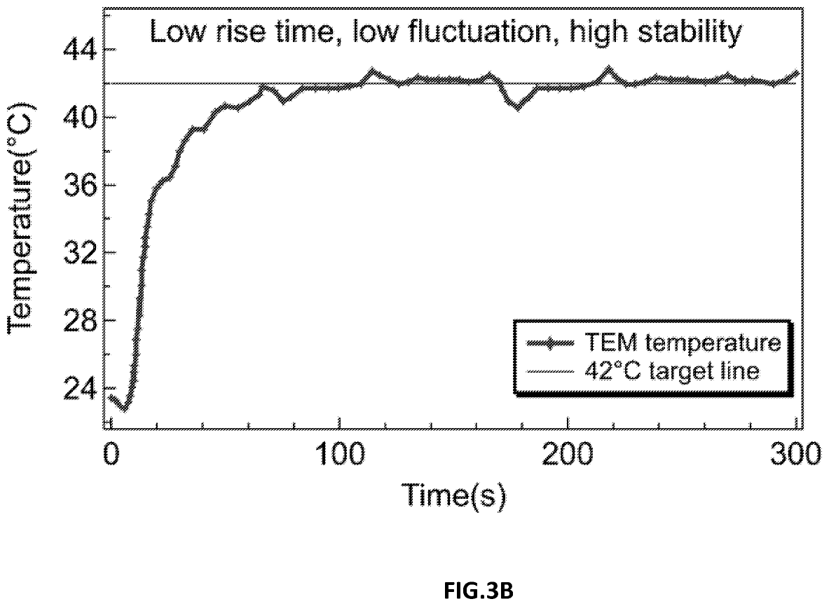

A simulation model is described in FIG. 3A. Therein, the temperature set point is the value subtracted from the measured value (feedback) from the temperature sensor 108 that is located in the heat-block. The difference (the error) is the input to the PID controller 140. The PID controller 140 calculates the next-step output value by using the error value with a set of parameters Kₚ, Kᵢ, and K_{d} (for proportional, integral, and derivative control respectively). The output is connected to the plant thermoelectric module (which is modeled by two main effects: thermal and Peltier). The output of the plant is read by the temperature sensor 108 and returned to the input.

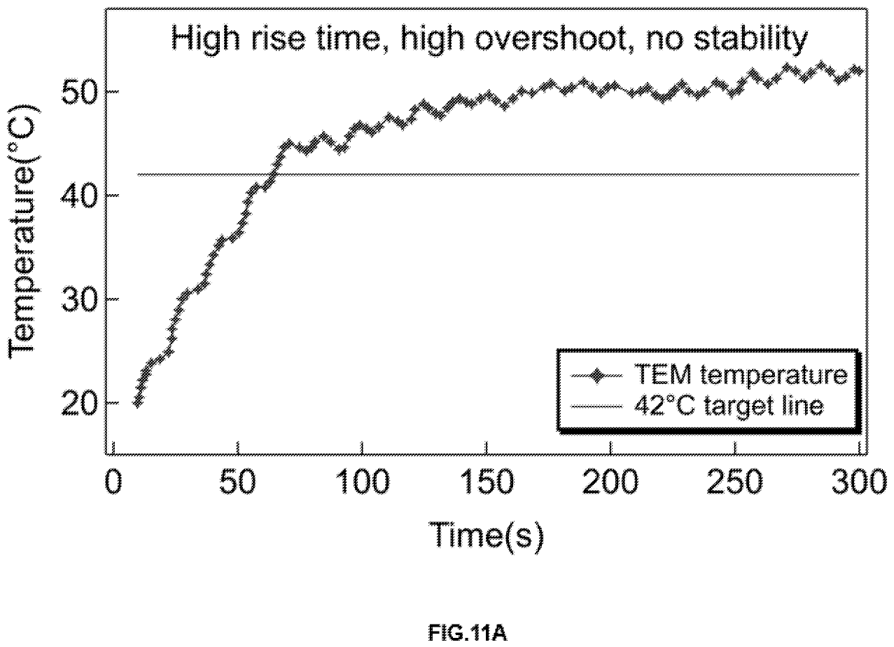

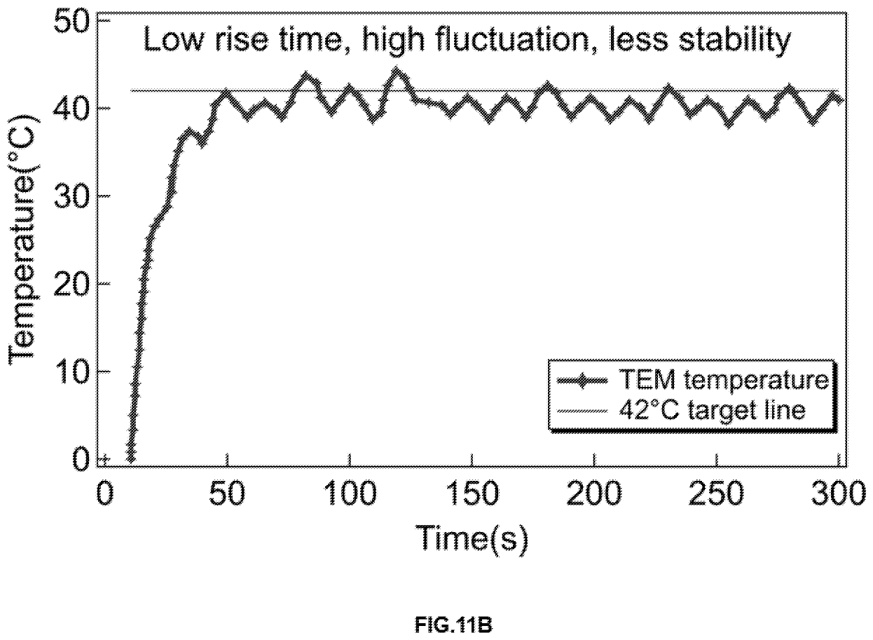

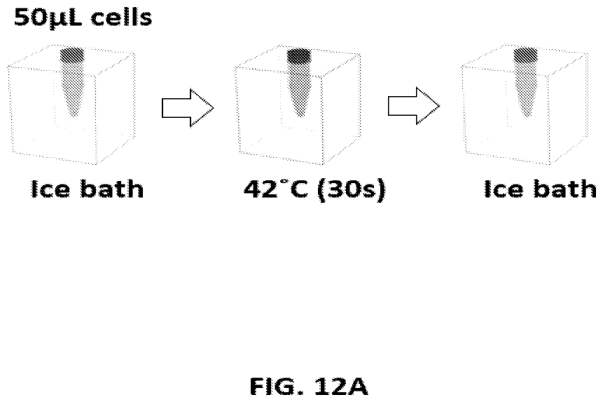

As a first test, the authenticity of the model was verified to check the robustness, stability, and accuracy of the closed-loop system. For that reason, three different scenarios were chosen to numerically simulate the target temperature of 42° C. Using parameters Kₚ=15, Kᵢ=1.2, and K_{d}=0.5 shows the optimal behaviour in terms of the demands (FIG. 3B, see FIG. 11 for other simulated scenarios)—i.e. fast rise time, low fluctuation, and high stability at the target temperature of 42° C. The simulation shows that parameters Kₚ and Kᵢ are sensitive to changing the stability and overshoot of the target. This observation is expected since increasing the proportional gain (Kₚ) will have the effect of reducing the rise time while the steady state error can be eliminated by introducing some element of integral control (Kᵢ). Given the simplicity of our model, this will allow users who are not familiar with control theory to implement closed-loop temperature control and integrate with reagent delivery control for digital microfluidics.

### Applications of “World-to-Chip” Interfaces

### Application #1: Bacterial Transformation

To evaluate the functionality of the world-to-chip interfaces, a bacterial transformation study was conducted on the digital microfluidic device 104. Bacterial transformation involves the insertion of plasmid DNA into E. coli (or some microbe) using a heat shock (or electric-field) based methods. Microbial plasmid-based systems can aid in the understanding of protein functions and interactions within the cell as well as serve as a storage system for genetic components. There has been much interest in automating the transformation procedure using digital microfluidics (or a derivative thereof)^{7, 33-34} since fields like synthetic biology typically requires the optimization of metabolic pathways³⁵ or building logic gated systems³⁶ consisting of many genetic elements that can be difficult to manipulate if manually performed. Here, a closed-loop temperature control and a reagent delivery system that is not present in these studies is disclosed.

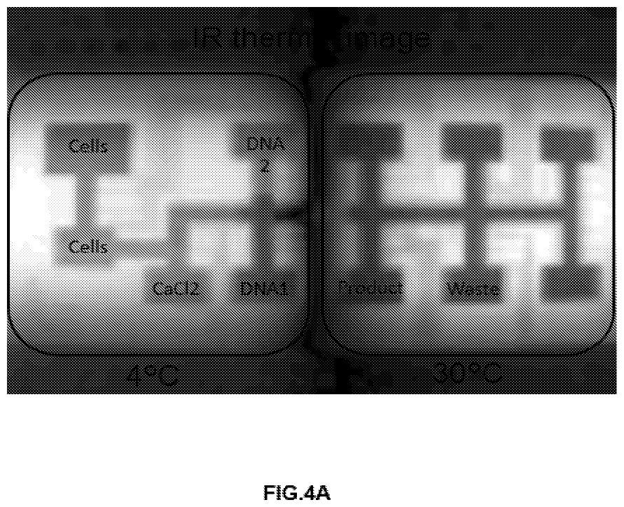

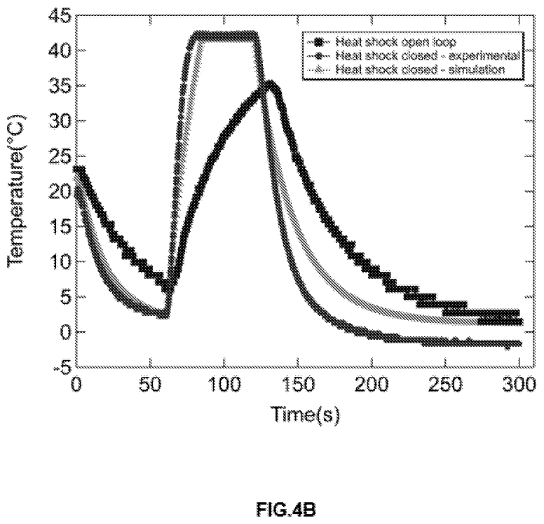

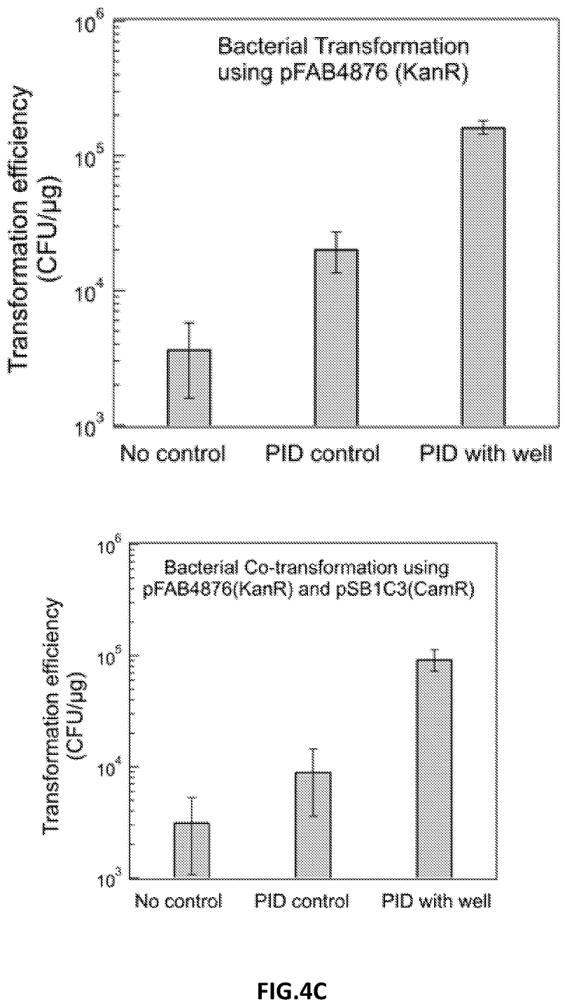

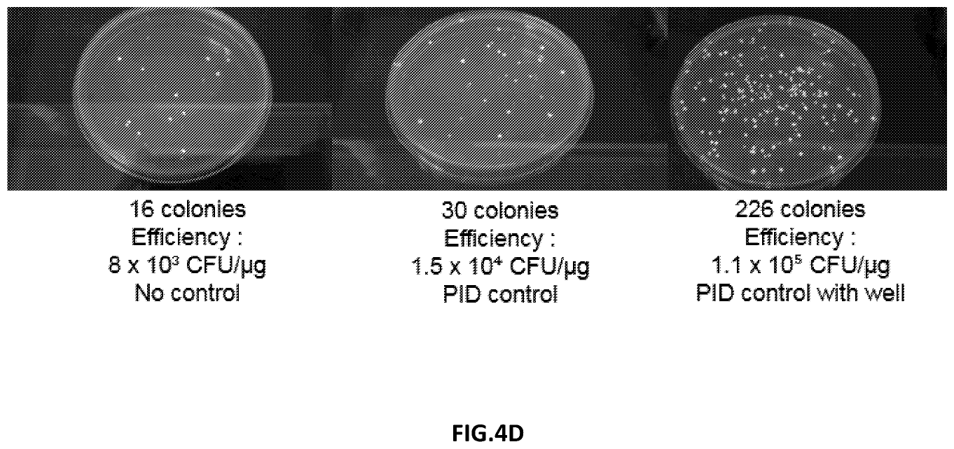

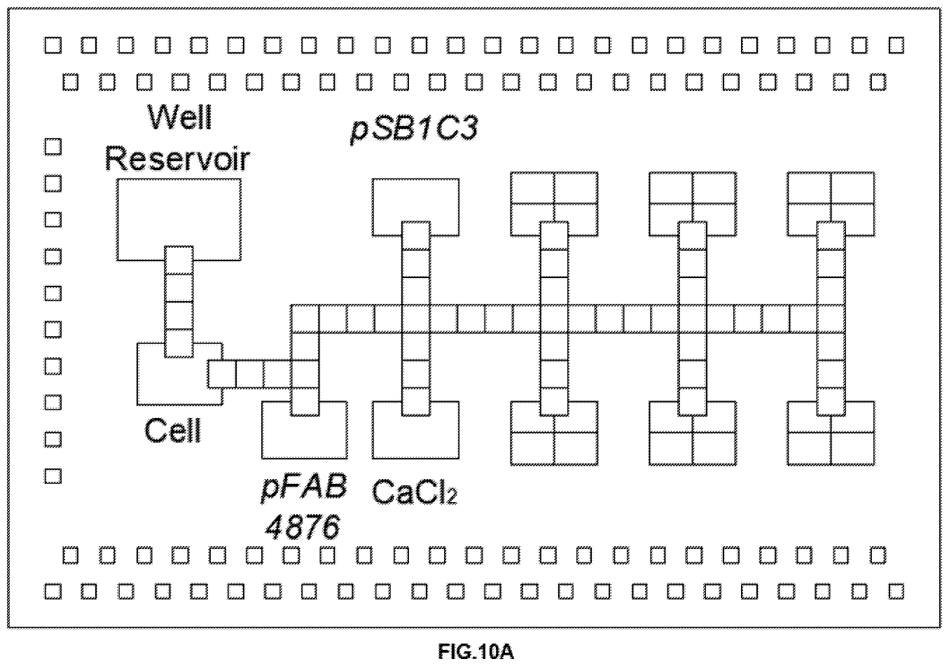

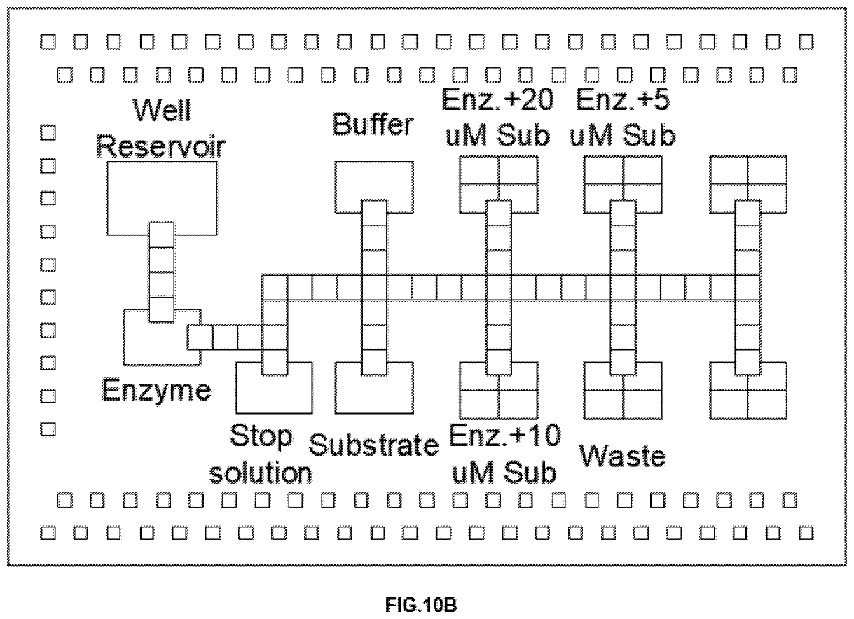

To perform transformation with the integrated world-to-chip interfaces described herein, a microfluidic device 1004 (see FIG. 10A) that is capable of automated transformation using heat-shock protocols was used. The device 1004 consists of four reservoirs 1050 that hold droplets of cells, LB media, CaCl₂) solution, and the plasmid coding for a fluorescent protein. Two new features (compared to other DMF and transformation studies) have been added to this configuration—(1) a 3D printed well 1030 that is used to automate reagent delivery to the reservoirs and (2) the closed-loop PID control of the TEC (not shown). The TEC module (not shown) below device 1004 was configured to provide a region with controllable thermal zones (see FIG. 4A). The canonical E. coli heat shock protocol requires temperature flux between 4° C. and 42° C. and the TEC was programmed to optimally control the fluctuation between these temperatures. An experiment was implemented to determine if the module was capable of reaching the desired temperatures. As shown in FIG. 4B, the open loop control of the TEC was not capable of moving between desired temperatures of 4° C. and 42° C. within the desired time of 1 min. However, with the closed loop PID control system, temperatures could quickly rise to 42° C. at a rate of 2.1° C./s and drop to 4° C. at a rate of 1.01° C./s (which was also verified via simulation). The closed loop PID system is important since it has been shown that temperature fluctuations affect transformation efficiency for some species of bacteria in which the length of incubation time at these temperatures can affect the uptake of the donor DNA.³⁷⁻³⁸

Next, the closed-loop control system was tested by experimentally transforming chemically competent DH5α E. coli cells with expressing GFP from the pFAB4876 plasmid. As shown in FIG. 4C and Table 1, the PID control TEC gave rise to an efficiency of 2.03×10⁴ colonies forming units per μg of DNA (CFU/μg). This was significantly (P=0.041 at 95% confidence) higher than the open-loop controlled transformation (3.67×10³ CFU/n). As expected, the temperature control for on-chip transformation played a significant role on the transformation efficiency (as observed in other studies⁸). In addition, with the reagent well delivery system, it may be possible to obtain higher transformation efficiency due to dispensing droplets at more precise volumes.

In three trials of conducting bacterial transformation on-chip using the reagent delivery system and with closed loop control, the transformation improved by 7-fold (p=1.48×10⁵ CFU/μg, p=0.007) compared to the closed-loop control and with usual dispensing techniques (i.e. no refilling the reservoir after one dispensing action). A possible explanation for this increase is that variable dispensing volumes can create variability in final DNA concentrations, pH of the solution, CaCl₂) treatment, and nutrient concentration in which all of these factors can affect overall transformation efficiencies.³⁹⁻⁴²

A third experiment was also conducted to measure the efficiency for transforming two DNA plasmids (i.e. co-transformation). Their wide-spread use in gene-editing⁴³ or metabolic engineering⁴⁴ that require transformation of multiple plasmids for the expression of multiple genes motivated this experiment. As shown in FIG. 4D and Table 1, the trends confirm to be similar such that the integration of both world-to-chip interfaces significantly improved the efficiency (p=0.001).

The differences between single and co-transformation (Table 1) and single transformation are generally higher in efficiency regardless of the methodology used for liquid delivery and temperature control.

### Application #2: Enzymatic Endoglucanase Assays

As a second demonstration of an application for our world-to-chip interfaces integrated with digital microfluidics, the implementation of enzymatic assays was explored. Enzymatic assays are often used to measure the activity of produced proteins and are vital to understanding enzyme kinetics and inhibition. The enzyme kinetics provides crucial information on the mechanism of the enzyme and on the interactions of the enzymes with substrates, inhibitors, drugs, etc. . . . . One of the fundamental applications of DMF is the implementation of enzymatic reactions.^{20, 45-48} The protocol typically consists of precise metering of reactants by dispensing droplets from reservoirs and merging and mixing to create a droplet that represents the microreactor. The microreactors on the DMF device have been analyzed using integrated in-line detectors^{19, 49-50} or with offline detectors (e.g., fluorescence-based plate readers)^{5, 14, 51}. However, most enzymatic assays on DMF are either performed at room temperature or without refilling of reservoirs for dispensing droplets.^{45, 52-53} These corresponding factors give rise to two limitations: (1) prevents the study of most industrial-based and thermo-resistant enzymes which are active at higher temperatures (>25° C.) and (2) prevents the study of different conditions on the device that requires droplets (containing substrate enzyme, or buffer) to be dispensed multiple times. These limitations motivate the development of our world-to-chip for digital microfluidics for the implementation of enzymatic assays that require heating and testing of multiple conditions in parallel.

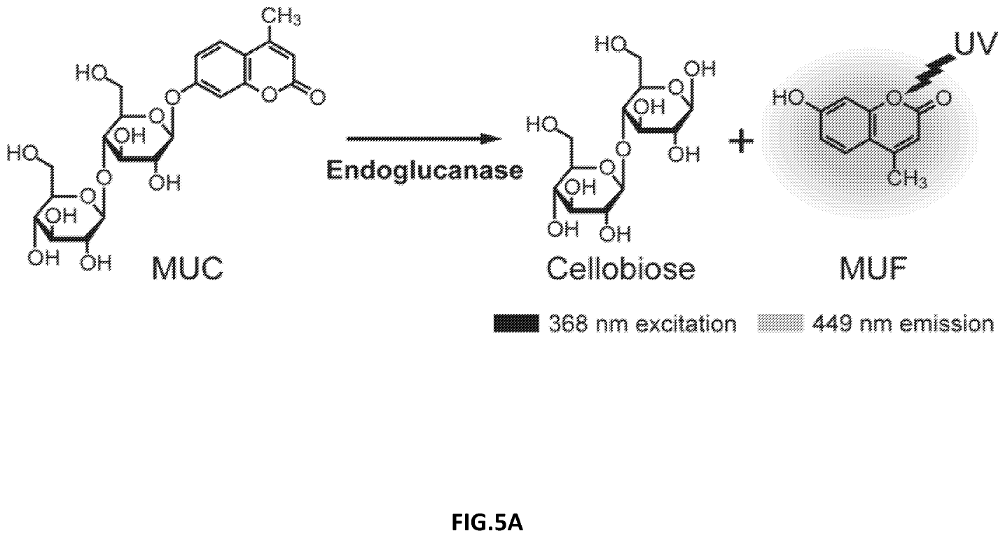

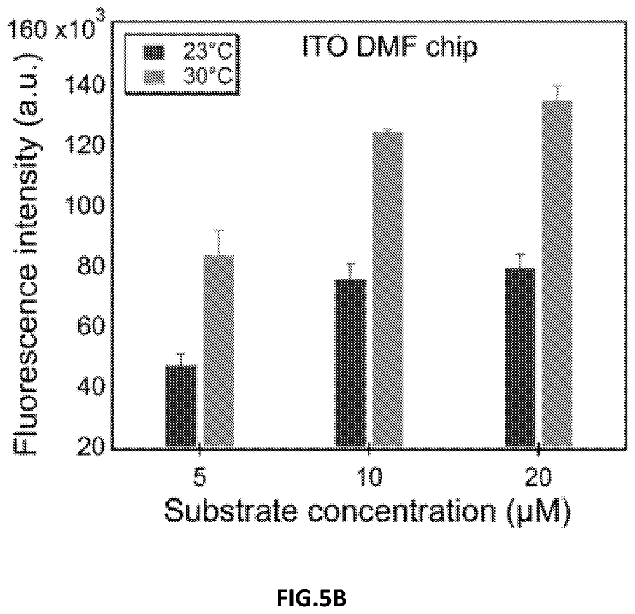

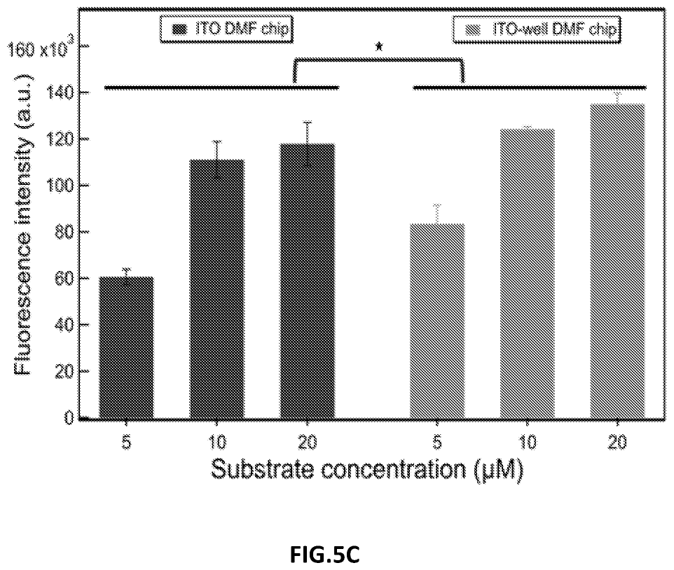

Herein, an enzymatic reaction that involves cellulase-based enzymes that are involved in the degradation of biomass into sugar that are useful for biofuel production was performed.⁵⁴ Specifically, an endoglucanase (which are optimal at higher temperatures) that is typically used to catalyze the hydrolysis of the (1,4)-glycosidic bonds was tested.⁵⁵ Droplets containing the 4-methylumbelliferyl β-D-cellobioside (MUC; substrate) and the endoglucanase enzyme were mixed and incubated for 30 min using the same DMF device as described abve. After incubating, the droplets were analyzed via fluorescence. FIG. 5A shows the chemical scheme of the assay which consists of using MUC in which the endoglucanase cleaves the β-(1,4) bond to give a cellobiose and a methylbelliferone fluorescence product. Using this chemical scheme, the enzymatic activity of the endoglucanase was tested using the 3D printed well for refilling of the reservoirs on the device to enable reproducible droplet dispensing of the enzyme and the closed-loop TEC to perform the assay at elevated temperatures (30° C.). The fluorescence output (i.e. the measure for enzyme activity) as a function of three substrate concentrations (5, 10, 20 μM) at two temperatures (23° C. and 30° C.), illustrated in FIG. 5B, show the key trend is reproduced—i.e. a higher temperature lead to higher output fluorescence compared to room temperature experiments.

In a second assay, the enzymatic assay was performed with and without the reagent delivery system and with the closed-loop TEC system set to a target temperature of 30° C. FIG. 5C shows the fluorescence output for each substrate concentration with and without the reagent delivery system. As shown, the fluorescence was significantly higher (ANOVA two-way test, P<0.05) for experiments with the reagent delivery system. A potential cause for this difference between the data are changes in the droplet volumes. There are studies that show small changes in volume can lead to changes in the pH, salt and detergent concentrations which can reduce the activity and stability of thermophilic enzymes by 20-30%.⁵⁶⁻⁵⁷

### Examples

Materials and Methods

Reagents and Materials

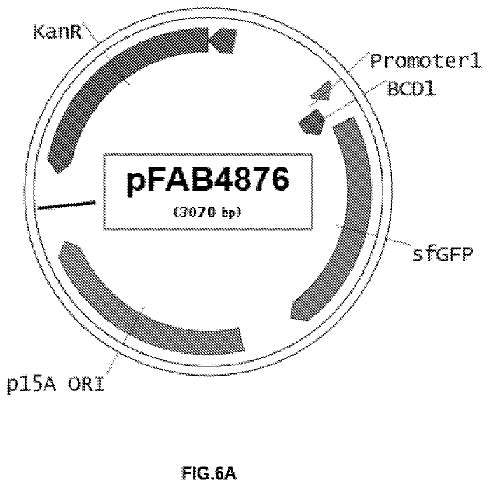

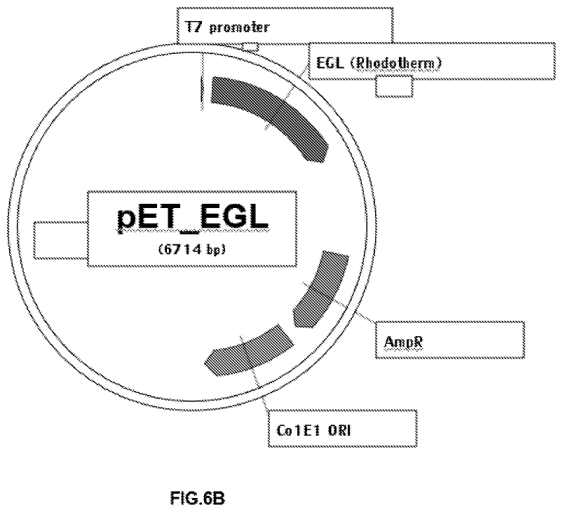

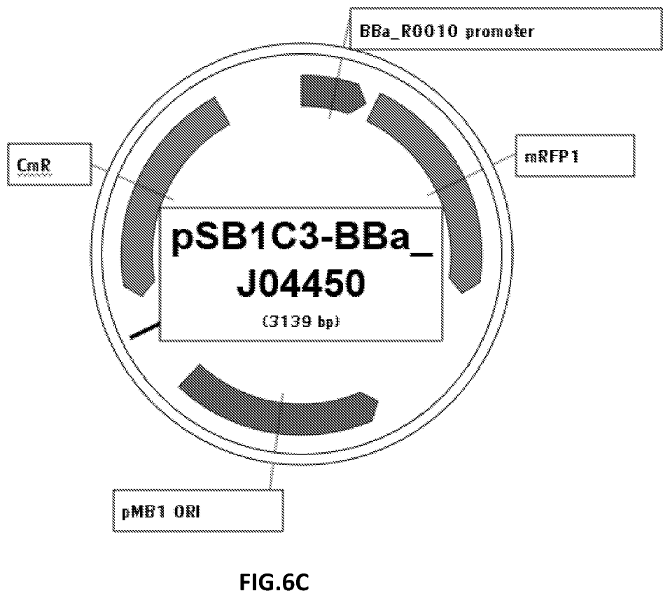

All general-use reagents were purchased from Sigma, unless specified otherwise. E. coli DH5α and pET16b vectors were generously donated by Dr. Vincent Martin and pFAB4876 plasmids were generously donated by the Joint BioEnergy Institute (see Table 1; FIG. 6 for plasmid maps). Mini-prep kits (cat no. BS413) were purchased from BioBasic (Amherst, N.Y.).

DMF device fabrication reagents and supplies included chromium with S1811 photoresist on glass slides from Telic (Valencia, Calif.), indium tin oxide (ITO)-coated glass slides, R_{S}=15-25Ω (cat no. CG-61IN-S207, Delta Technologies, Loveland Colo.), FluoroPel PFC1601V from Cytonix LLC (Beltsville, Md.), MF-321 positive photoresist developer from Rohm and Haas (Marlborough, Mass.), CR-4 chromium etchant from OM Group (Cleveland, Ohio), and AZ-300T photoresist stripper from AZ Electronic Materials (Somerville, N.J.). TEC/Peltier module from TE technology (Traverse City, Mich., USA). For 3D printing, polylactic acid (PLA) material were purchased from 3Dshop (Mississauga, ON, Canada). 3-(Trimethoxysilyl) propyl methacrylate solution for silanization from Sigma (cat no. 440159). Parylene C pellets were purchased from Speciality Coating Systems (Indianapolis, Ind.).

Designing, Fabrication, and Operation of the 3D Printed Well

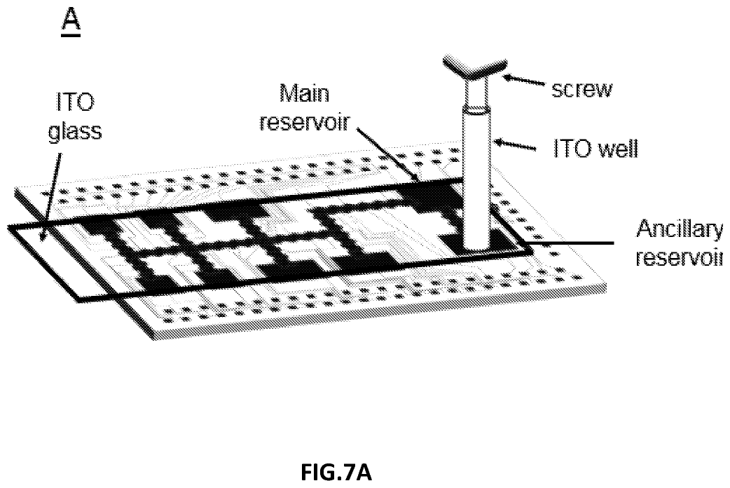

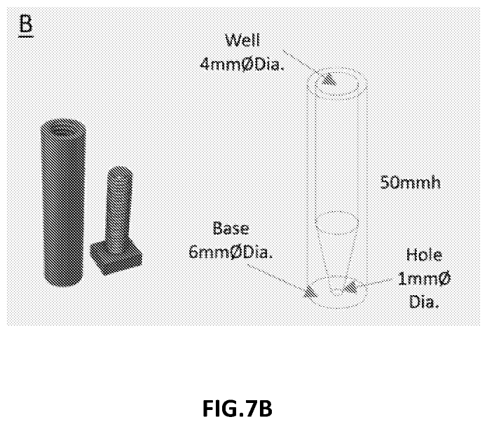

3D printed wells were designed using Fusion 360 3D CAD/CAM design software (Autodesk, CA United States). The design was printed using the Ultimaker 2+ extended (Shop3D, Mississauga, ON, Canada). All cylindrical wells were printed on polylactic acid (PLA) with an outer diameter of 8 mm and with a height of 45 mm and a 1 mm diameter hole (see FIG. 7). For 3D printing, STL files were generated and converted to gcode format using Acura 3D conversion with the following settings: nozzle—0.4 mm, material—PLA, and profile—high quality. In the print setup settings, the recommended button was checked, the infill parameter was checked as ‘Dense’ and the helper parameter checked as ‘Print Build Plate Adhesion and Print Support Structure’.

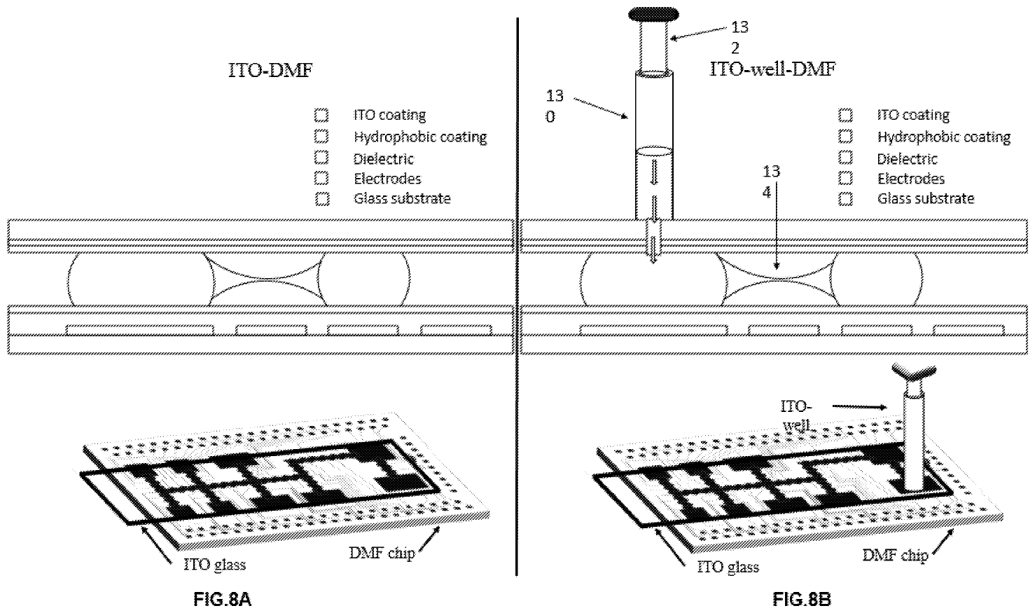

To use the well 130 as a reagent delivery system, the well was aligned directly to the drilled hole on the ITO top plate which may be called ‘ITO-well-DMF’ configuration (FIG. 8). About 400 μL of fluid was pipetted into the well and a 3D printed screw 132 was used to control the delivery of the fluids into the reservoir 134 on device 104. The device 104 consisted of at least two reservoirs—a main reservoir 134 for active dispensing of a unit droplet and an ancillary reservoir 135 for refilling the main reservoir 134. The well was aligned to the center of the ancillary reservoir 135. The operations of refilling the main reservoir 134 proceeded in the following four steps: (1) liquid from the 3D printed well was delivered to the ancillary reservoir 135 using the screw via rotation action, (2) a liquid neck from the ancillary reservoir 134 is actuated to the main reservoir 134, (3) the volume in the main reservoir 134 is measured via impedance by applying 160 V_{RMS} to the electrode (see circuit FIG. 9A and calibration curve FIG. 9B), and (4) when the volume in the main reservoir 134 reached 7 μL, the main 134 and ancillary 135 reservoirs are activated to initiate a splitting operation (i.e. breaking off the liquid neck between the two reservoirs). These four steps for refilling a reservoir is repeated for every droplet being actively dispensed from the main reservoir 134. For dispensing experiments on ITO-DMF (i.e. no well on the top plate) and ITO-well-DMF configurations, droplets were dispensed from the main reservoir 134 and the reservoir and dispensed droplet volumes were measured by impedance (FIG. 9).

TEC Design, Operation, and Simulation

A 20×40 mm TEC (TE technology INC, Texas, USA) was integrated below the DMF device that was used to provide cooling (4° C.) and heating (42° C.) temperatures for procedures related to on-chip transformation. The TEC was integrated into a 3D printed module with four 3D printed screws that can be interfaced with the DMF device 104 (FIG. 1). A 20×40×5 mm aluminium heat block with a 1.5 mm drilled hole at the center was situated between the TEC and the DMF chip. The control hardware circuit for changing the temperature consisted of an Arduino microcontroller (Arduino Uno, Italy), a driver motor (consisting of a two half-bridge driver chip and a low resistance N-channel MOSFET) (Amazon, Mississauga, ON, Canada), and a resistance temperature detector (Building Automation Products, Inc., Gays Mills, WS) that was placed inside a hole of the aluminum heat block. The hole was filled and sealed with thermo-paste (GC electronics, Rockford, Ill.) to secure the temperature detector in place. Finally, the bottom of the aluminum heat block consisted of a 12 V DC cooling fan used to dissipate the excess heat produced by the TEC while supplying a temperature of 4° C. For open-loop control, the TEC was connected to a DC power supply through a MOSFET dual H-bridge driver. The driving current was controlled by an Arduino Uno microcontroller programmed by the user. Generally, the TEC was set to operate in 4° C. for 60 s, then was rapidly increased to 42° C. for 60 s followed by a decrease to 4° C. for 120 s.

For closed-loop control, the temperature of the system was stabilized by a negative control-loop feedback system via the microcontroller. A proportional-integral-derivative (PID) based software code (see Supplementary Information for simulation modules) was written in Matlab/Simulink (Mathworks, MA USA). Simulations of the temperature control were executed by changing rise time, over shoot, settling-time, and steady-state error values as shown in equation 1:

$$u ( t ) = {K}_{p} e ( t ) + {K}_{i} {\int }_{0}^{t} e ( {t}^{′} ) {dt}^{′} + {K}_{d} \frac{{d}_{e} ( t )}{dt} \qquad Eq . ( 1 )$$

where, Kₚ, Kᵢ and K_{d} represent gain coefficients for the proportional, integral, and derivative terms respectively, e(t) represent the error between the setpoint value and the sensor measured value parameter, and u(t) was a controller output. In the plant simulations, the TEC was modeled as a thermodynamic system.²¹ The thermodynamic model was split into five main energy processes: thermal conduction, Joule heating, the Peltier cooling/heating effect, the Seebeck effect, and the heat transfer effects. These processes were modeled by four equations (see Table 2) and parameters were taken from the manufacturer datasheet.
Microscale Bacterial (co-) Transformation

Prior to the experiment, the competent E. coli cells were thawed on ice for 10 min. Plasmid DNA (pFAB4876 and pSB1C3) was diluted to a concentration of 1 μg/μl. CaCl₂) stock was prepared at 150 mM to maintain final concentration of transformation solution on chip at 75 mM of CaCl₂). For on-chip transformation, two TEMs 124 were attached to the bottom of the DMF chip that provided two controllable thermal zones (4° C. and 42° C.) and all reagents were initially pipetted to the cold region on the chip.

For ITO-DMF configuration, 7 μL of each reagent including competent cells, pFAB4876, pSB1C3, and CaCl₂) were pipetted into the corresponding reservoir. A premade sequence code which was using in-house software was executed to apply a voltage of 160 Vᵣₘₛ at 15 kHz frequency to dispense a 1 μL droplet from the reservoir. Dispensed droplets (1 μL each) containing the DNA plasmid, E. coli cells in LB media and CaCl₂) were merged together on the chip in an equal volume ratio for single (or co-) transformation experiments. After merging and mixing, a heat shock protocol was applied which consisted of 60 s at 42° C. followed by 3 min at 4° C. The solution was taken out by pipette and placed into a micro centrifuge tube. 100 μL of fresh LB were added to the mixture and then the tube was placed at the incubator at 37° C., 220 rpm for 1 h recovery. The transformed cells were plated on an agar plate with kanamycin (50 μg/mL) or kanamycin and chloroamphenicol (35 μg/mL) antibiotic selection overnight for single and co-transformation studies respectively. Three replicates were conducted in parallel on the same device.

For ITO-well-DMF configuration, 400 μL of cells were added to the 3D printed well (instead of directly in a reservoir) and 7 μL of the DNA plasmids and CaCl₂ were added directly to the reservoirs on chip. The procedures followed the ITO-well transformation protocol with an additional four steps to refill the main reservoir that contained cells. Briefly, to refill the reservoir, the solution containing cells were delivered to the ancillary reservoir 135 by rotating the screw to descend further into the 3D printed well to push the solution from the well to the ancillary reservoir 135. Next, the fluid was actuated from the ancillary reservoir 135 to form a liquid neck which was combined with the liquid in the main reservoir. A splitting step occurred to break off the liquid neck after filling the main reservoir to a desired volume of 7 μL (which was measured by impedance). Three parallel replicate measurements were performed on one device.

### Enzymatic Endoglucanase Assay

The endoglucanase enzyme assay was carried out on one of the two configurations ITO-DMF and ITO-well-DMF, and at room temperature or 30° C. For the 30° C. condition, the TEM 124 was integrated below the assay areas and a closed-loop PID control was enabled to maintain a steady-state temperature. On the ITO-DMF configuration, a unit droplet of enzyme solution (˜1 μL) was dispensed into one of three assay areas on the device using a voltage of 160 V_{RMS} at 15 kHz. A substrate solution containing 40 μM 4-methylumbelliferyl β-D-cellobioside (MUC) in buffer (50 mM sodium-phosphate, pH 7.0) was dispensed from the reservoir and stored in an assay area. A second unit droplet of substrate solution was dispensed and serially diluted to 20 and 10 μM MUC droplets with buffer solution by mixing and splitting droplets. Two droplets containing 20 and 10 μM MUC were then individually stored in an assay area. To start the reaction, all three substrate-containing droplets were simultaneously mixed with the enzyme droplets in the assay areas (if required, the TEM 124 was activated). After 30 minutes of incubation, a unit droplet of quenching solution (0.3 M glycine-NaOH, pH 11.0) was mixed with each reaction droplet in the assay area. On the ITO-well-DMF configuration, the same droplet operations were carried out, except that the well was used to replenish the main reservoir that contained enzyme after actively dispensing an unit droplet. After the assay, the device was placed on top of a 96 well-plate and into a CLARIOStar plate reader (BMG Labtech, Ortenberg, Germany) to measure 4-methylumbelliferone (MUF) fluorescence at 449 nm with 368 nm excitation. The fluorescence intensity was measured by using the well-scanning function (scan matrix=15×15, scan diameter=6 mm, focal height=4.0 mm and gain=1180), and the maximum fluorescence intensity value for each droplet was recorded for analysis. Each assay was repeated three times. All solutions used on the device contained 0.05% F-68 Pluronics additive.

### Microfluidic Device Fabrication and Operation

Devices were designed using AutoCAD 2017 (Autodesk, San Rafael, Calif.) and fabricated in the Concordia cleanroom. The DMF fabrication procedure followed a previous protocol¹⁻² using high resolution 25,400 dpi transparency masks printed by CAD/Art (Bandon, Oreg.) services. Briefly, glass substrates pre-coated with S1811 photoresist (Telic, Valencia, Calif.) were exposed to UV for 8 s on a Quintel Q-4000 mask aligner (Neutronix Quintel, Morgan Hill, Calif.) to imprint the transparency masks design. These were developed in MF-321 for 2 min with shaking and rinsing with DI water. Developed slides were then baked at 115° C. for 1 min before etching in CR-4 chromium etchant until the pattern was clearly visible. The remaining photoresist was then removed in AZ-300T stripper for 2 min. After rinsing with DI water and drying, a silane solution comprising de-ionized water, 2-propanol and (trimethoxysilyl)-propyl methacrylate (50:50:1 v/v) was added to the devices in a Pyrex dish for 15 min. Devices were primed for dielectric coating with 15 g of Parylene-C (7.2 μm) in a SCS Labcoater 2 PDS 2010 (Specialty Coating Systems, Indianapolis, Ind.), and coated with Fluoropel PFC1601V (Cytonix, Beltsville, Md.) in a Laurell spin coater (North Wales, Pa.) set to 1500 rpm for 30 s with 500 rpm/s acceleration followed by 10 min baking at 180° C.

Prior to experiments, two types of top-plates were prepared—ITO-DMF or ITO-well-DMF (FIG. 8). ITO-DMF were coated with FluoroPel PFC 1601V by spin coating and then post-baked as described above. For ITO-well-DMF, 1 mm diameter holes were drilled on the top plate using a Micromill (Proxxon MF 70, S.A. Wecker, Luxemburg) with a diamond drill bit (dia. 1.00 mm, L=38 mm, diamond tip length: 3.50 mm, shank: 0.70 mm; ordered from KLY Amazon). These were diced into 25 mm×75 mm pieces and were coated with Fluoropel PFC1601V and post-baked using the conditions described above on both sides. A 3D-printed well (with 1 mm diameter holes) that is coated inside with Fluoropel (using a cotton swab) were aligned directly on top of the holes on the ITO and glued (LePage super glue, Mississauga, Ontario) to the glass and dried for 5 min at room temperature. Both of these ITOs were joined to the bottom substrate with two pieces of double sided tape, resulting in an inter-plate gap of ˜140 μM.

A custom-designed fabricated device was used in this study for both bacterial transformation and enzymatic assay. After combining the top and bottom plates, the DMF device was primed to be interfaced through our automation system (see our online GitHub repository: [https://github.com/shihmicrolab/Automation]) that will automate the droplet movement on the devices. Electrodes were actuated using 160 V_{RMS} at 15 kHz using solid-state relays controlled by in-lab software.

Competent Cell Preparation

Prior to preparation, a −80° C. freezer stock of DH5α was streaked-out on a plate containing lysogeny Broth (LB) and 8% agar and grown overnight (12 to 16 h). On day 1, a single bacterial colony (2 to 3 mm in dia.) (from the streaked plate using the −80° C. stock) was inoculated in 5 mL of LB in a 20 mL flask overnight (about 12 to 14 h) at 37° C., 220 rpm. On day 2, 6 mL of the culture was added into 600 mL of fresh LB medium and incubated in 37° C. at 220 rpm. After 1.5 h, the OD 600 was measured every 15 min until the OD 600 level reached 0.45. The cells were transferred to cold 50 mL centrifuge tubes. Before spinning, the centrifuge was spun for a few seconds to reach 4° C. Then the cells were recovered by centrifugation at 1000 g for 10 min at 4° C. after which the medium was decanted from the cell pellets. The bacterial sediment was re-suspended in 50 mL of ice-cold solution containing 15% glycerol with 75 mM of CaCl₂) and was incubated on ice for 5 min and then centrifuged at 1000 g for 10 min. After centrifugation, the cells were re-suspended in 2 mL of 75 mM CaCl₂) with 15% glycerol solution. 100 μL aliquots were snap frozen in liquid nitrogen and stored in −80° C. freezer.

Cloning and Protein Expression

The sequence for the Rhodothermus marinus SG0.5JP17-172 endoglucanase gene (EGL) was obtained from NCBI (GenBank accession number WP_014065767.1) and was synthesized by IDT (Coralville, Iowa) as a linear DNA fragment. The gene was amplified by PCR using Phusion polymerase (Thermo Fisher scientific, Waltham, Mass.) according to manufacturer's instructions (initial 98° C. for 30 s, 98° C. for 10 s, 55° C. for 30 s, 72° C. for 30 s/kb, final 72° C. for 10 min and 4° C. on hold, with 35 cycles of amplification). The following primers were used to introduce a 5′ XbaI and a 3′ BamHI restrictions sites:

Forward:
5′-TGACTGACTCTAGAAATAATTTTGTTTAACTTTAAGAAGGAGATATA
CCATGCGTGTATTGGCGCTGC-3′
Reverse:
5′-GCATGCATGGATCCCTAATTGCGTGGATTTAATTGGCGC-3′

The PCR product was purified, digested with XbaI and BamHI for two hours, and ligated into a linearized pET16b vector. The ligation product was transformed into chemically competent E. coli DH5a cells and plated on selective media. Single colonies were inoculated in 5 mL of LB media containing 100 μg/mL ampicillin overnight and plasmids were extracted using a BioBasic miniprep kit. Proper insertion of the gene was verified by digesting 2 μg of plasmid with XbaI and BamHI checking proper insert size on a 0.8% agarose gel. The cloned plasmid (pET_EGL) was transformed into E. coli BL21(DE3) for protein expression. The transformed cells were inoculated overnight in a 5 mL pre-culture. The next day, a 100 mL starter culture of low optical density (OD) was generated by diluting the overnight culture and grown at 37° C. with 200 rpm shaking. Upon reaching OD 0.4, expression of the EGL gene was induced by addition of 1 mM IPTG for 6 hours. The final induced culture was divided into 40 mL aliquots and centrifuged at 4000 rpm for 5 min. The pellets were frozen and kept at −20° C. for later used. Thawed pellets were lysed at 4° C. for 15 min by resuspending in 10 mL of lysis solution comprising 1 mg/mL lysozyme, 25 U/ml benzonase and 1 mM phenylmethanesulfonylfluoride (PMSF). The lysates were centrifuged again at 4000 rpm for 5 min to collect the protein pellets. The pellets were resuspended in 10 mL of assay buffer (50 mM sodium-phosphate, pH 7.0) and diluted 50-fold in the same buffer before use.

TABLE 1
Description of strains and plasmids used in this study

| | | |
|---|---|---|
| Strain or | | |
| plasmid | Relevant genotype and description | Ref. or source |

Strains

| | | |
|---|---|---|
| E. coli | fhuA2 Δ(argF-lacZ) U169 phoA | Vincent Martin Lab, |
| DH5α | glnV44 Φ80 Δ(lacZ)M15 gyrA96 | Concordia University |
| | recA1 relA1 endA1 thi-1 hsdR17 | |
| E. coli | F-ompT gal dcm lon hsdSB(rB- | Vincent Martin Lab, |
| BL21 | mB-) λ(DE3 [lacI lacUV5-T7 | Concordia University |
| | gene 1 ind1 sam7 nin5]) | |

Plasmids

| | (origin, resistance, gene of interest?) | |
|---|---|---|
| pFAB | DH5α, Kanamycin, GFP | Vincent Martin Lab, |
| 4876 | | Concordia University |
| pSB1C3 | DH5α, Chloramphenicol, RFP | Vincent Martin Lab, |
| | | Concordia University |
| pET-EGL | BL21 (DE3), Ampicillin, EGL | This study |

TABLE 2
Equations and parameters used to model the closed-loop
thermoelectric cooler.

| Process | Equation | Parameter |
|---|---|---|
| Thermal conduction | Qₜₕ = −ΔT * Kₜₕ | Kₜₕ: Thermal conductivity coefficient |
| | | of TEC = 0.39 |
| Joule heating | Qⱼ = I²R | R: TEC internal resistance = 1.4Ω |
| | | I: drawn current |
| Peltier | Qₚₐ = Sₘ * I * Tₕ | Sₘ: Seebeck coefficient = 0.018, |
| cooling/heating | Qₚₑ = Sₘ * I * T_{c} | I: drawn current, |
| | | Tₕ hot side temperature (K), |
| | | T_{c}: cold side temperature (K) |
| Seebeck | Vₛ = Sₘ * ΔT (Tₕ-T_{c}) | Sₘ: Seebeck coefficient = 0.018, |
| | | Tₕ: hot side temperature (K), |
| | | T_{c}: cold side temperature (K) |
| Heat transfer | Qₜₐ = Qₚₐ − 0.5Qⱼ − Qₜₕ | Qₚₐ: Peltier heating, |
| equation in TEC | Qₜₑ = Qₚₑ + 0.5Qⱼ − Qₜₕ | Qₜₐ: Peltier cooling, |
| | | Qₜₕ: Thermal conduction, |
| Aluminum heat block temperature | ${T}_{h} = \frac{\int ( ( {T}_{amb} * {K}_{th} ) + {Q}_{te} )}{Alum . thermal mass}$ | Qₜₑ: total heat transfer (hot side), Kₜₕ: thermal conductivity of Aluminum = 0.03, T_{amb}: room temperature in ° C., Aluminum thermal mass = 12.776 |
| Heat sink block temperature | ${T}_{c} = \frac{\int ( ( {T}_{amb} * {K}_{th} ) + {Q}_{ta} )}{Heatsink thermal mass}$ | Qₜₐ: total heat transfer (cold side), Kₜₕ: thermal conductivity of metal = 10, Heatsink thermal mass = 200 |

TABLE 3
Comparison of “world-to-chip” interfaces on DMF and our work

| Property | Current literature | Our work |
|---|---|---|
| Reagent | Use of pressure sources | No external pressure |
| Delivery | and external moving | sources, only a plunger |
| | parts to deliver liquid | mated to a 3D printed well |
| | No refilling in reservoirs | Refill reservoirs after every |
| | Typical volumes ~1-2 μL | dispensing action resulting |
| | References: ³⁻⁶ | in reproducible dispensing |
| | | Can store at least 400 μL of |
| | | volume in the well |
| Tem- | Off-chip heating^{2, 7} | On-chip heating and cooling |
| perature | Directly fabricate | Interfacing TEC directly |
| Control | electrode heaters | below device-no extra |
| | on device ⁸⁻⁹ | fabrication needed |
| | Open-loop control¹⁰-no | Closed-loop control with |
| | tuning or simulation | modeling of the system |
| | provided | enabling rapid changes in |
| | | temperature |

Thermal Plant

To simulate a closed-loop system, the PID controller may be modelled using the equations shown below. The thermal plant was modeled as a thermodynamic system (similar to ref⁷⁷) consisting of two components: the thermoelectric module (TEM) and heat block modules. The TEM has the form of thermopiles that are connected electrically in series to increase the operating voltage and thermally in parallel to decrease the thermal resistance. It is also sandwiched between two ceramic plates for uniform thermal expansion. There are four main energy processes taking place in the TEM pellets:

Thermal conductivity

Joule heating

The Peltier cooling/heating effect and

The Seebeck effect

### Thermoelectric Module (TEM) Simulink Simulation

Thermal Conductivity

The phenomenon of thermal conduction is a Fourier process that is described by the thermal conductivity Ki of the material. Both ceramic plates and interconnected metals have high thermal conductivity to ensure uniform temperature at either end. Neglecting the contribution of the metal interconnectors and ceramic plates, the analysis of a TEM can be conducted by analyzing a single pellet or thermocouple without loss of generality. Hence the analysis of N thermocouple is the same as analysis of one thermocouple, the heat transfer of thermal conduction is described by:

Qₜₕ=−ΔT×Kₜₕ

where Kₜₕ is thermal conductivity coefficient

Joule Heating

Joule heating is a physical process of heat dissipation in a resistance element. The flow of electric current through the TEM will additionally cause resistive heating of the thermocouples. The total Joule heat dissipated in TEM is:

Qⱼ=I²R

where R is the TEC internal resistance, I, is current drawn from DC power supply, and Qj is the calculated power (heat produced by passing current). Irrespective of the temperature gradient, Joule heating can be considered as equally divided between the two sides of the TEM.

Peltier Cooling/Heating Effect

The Peltier cooling/heat effect is a phenomenon of heat absorption/dissipation by a junction between two dissimilar materials when electrical current flows through the junction. The absorbed/emitted heat of an N-couple TEM is:

Qₚₐ=Sₘ*I*Tₕ

Qₚₑ=Sₘ*I*T_{c}

where Sm is the Seebeck coefficient, I, is the current drawn from DC power supply, Th is the hot side temperature in Kelvin, Tc is the cold side temperature in Kelvin, Q is the calculated power (heat produced by passing current). For calculation of emitted heat, the cold side temperature should be used and for the absorbed heat, the hot side temperature should be used.

### Seebeck Effect

When a temperature gradient is imposed on a conductor under an open-circuit condition, the creation of an electrical potential difference between the hot and cool sides of the conductor is called the Seebeck effect. The generated Seebeck voltage, called the back electromotive force (BEMF), in a TEM is expressed as:

Vₛ=Sₘ*ΔT

where Sₘ is the Seebeck coefficient. Now the total heat transfer at two side of Peltier equals to:

Qₜₐ=Qₚₐ−0.5Qⱼ−Qₜₕ

Qₜₑ=Qₚₑ+0.5Qⱼ−Qₜₕ

The Peltier output voltage is:

Vₜ=Vₛ+IR

where Qₜₑ is the total heat transfer in the hot side calculated in TEM, Kₜₕ is the thermal conductivity of Aluminum, T_{amb} is the room temperature in ° C., and thermal mass can be calculated based on the heat block dimension and thermal capacity coefficient.

### Heatsink Block Temperature

The temperature of heatsink block connected to the fan is calculated as follows:

$${T}_{c} = \frac{\int ( ( {T}_{a m b} * {K}_{t h} ) + {Q}_{ta} )}{Heatsink thermal mass}$$

where Qₜₐ is the total heat transfer in cold side calculated in TEC, Kₜₕ is the thermal conductivity of metal, T_{amb} is the room temperature in ° C., and Heatsink thermal mass can be calculated easily for the heatsink.

Further to the above, it can be noted that performing transformation of DNA in the microscale requires high efficiencies. Previous studies have resorted to electroporation on device which promises high efficiencies but are difficult and expensive devices to manufacture. Preparing chemically competent cells for routine cloning remains the standard in most laboratories. Given this, optimizing DMF for use of chemically competent cells is an attractive pursuit. Herein, a heat shock transformation method for chemically competent E. coli which uses function mapping PID control to optimize temperature conditions based on an analytical model and thermal dynamic simulations is also described. This methodology may improve on previous on-chip transformation and helps better understand the thermal system of heat shock transformation.

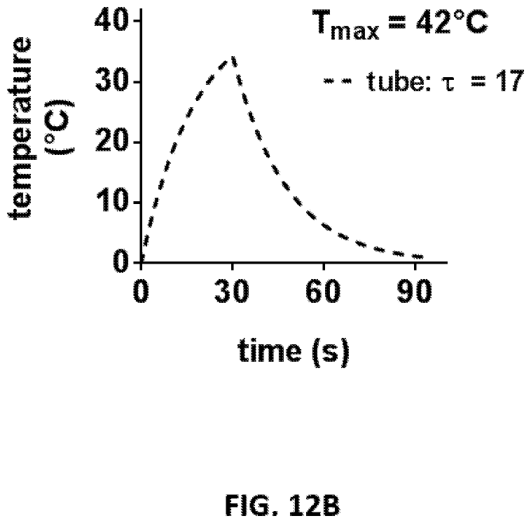

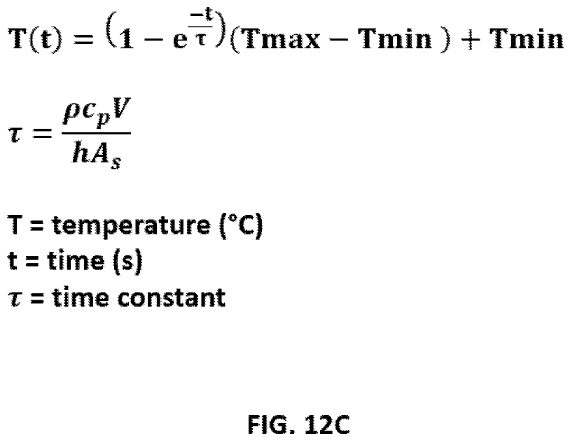

The first stage of the method is to establish the temperature system involved for heat shock of chemically competent E. coli cells (see FIG. 12A). This may be done using 1.5 mL microcentrifuge tubes filled with 50 μL of chemically competent E. coli and 10 ng of DNA transferred between an ice bath and warm water baths set to 42° C. for 30 sec (see FIGS. 12A and 12B), according to at least one embodiment. The transfer function for temperature in the liquid of the tubes can be simplified by the equation shown in FIG. 12C. The time constant (τ) is an important factor.

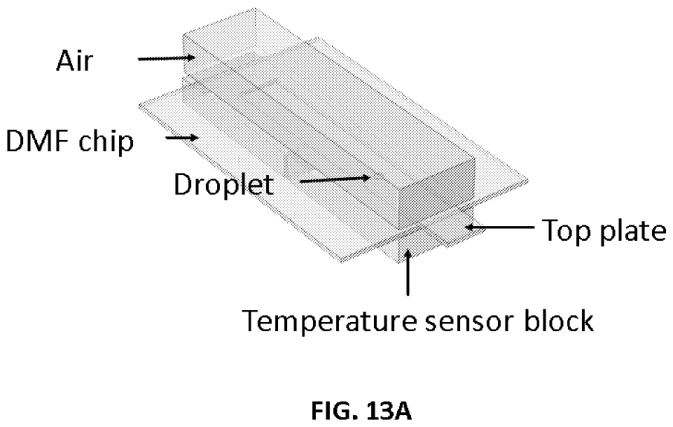

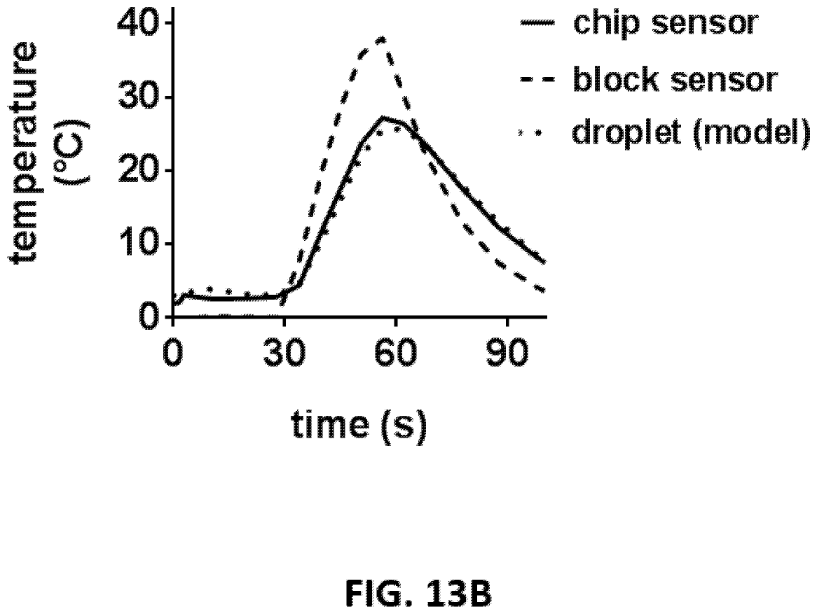

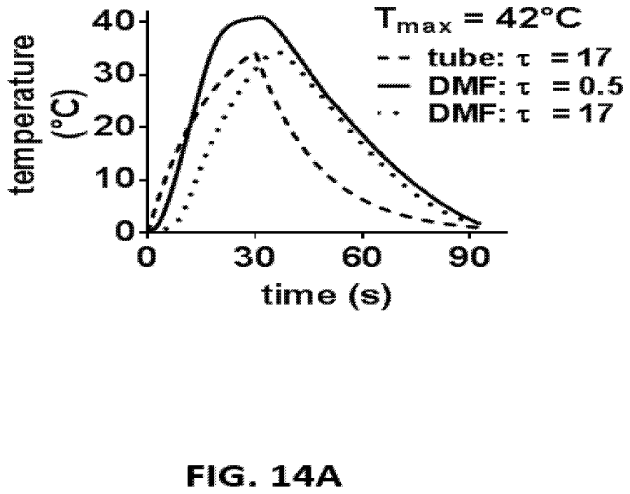

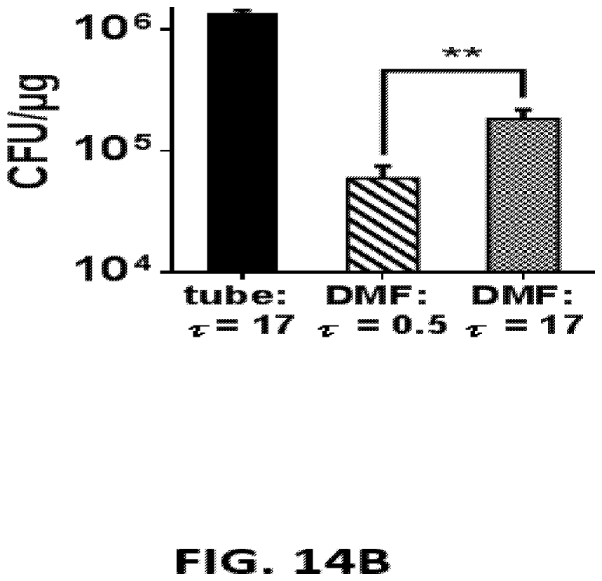

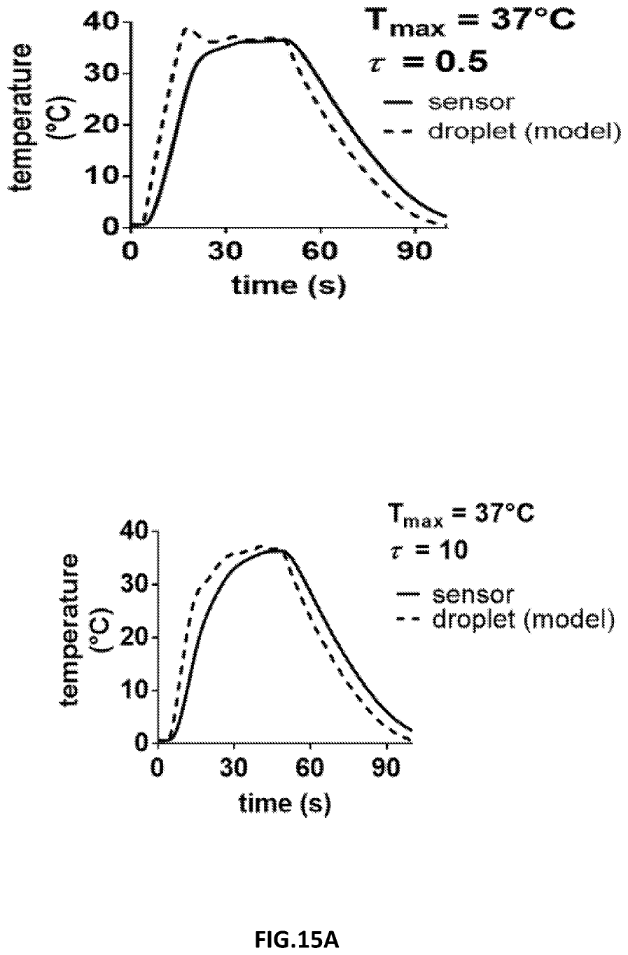

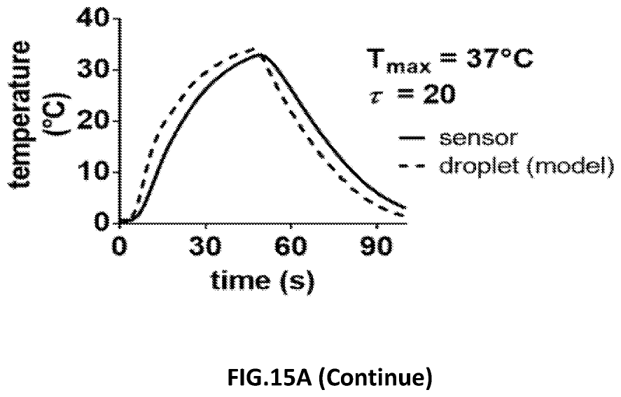

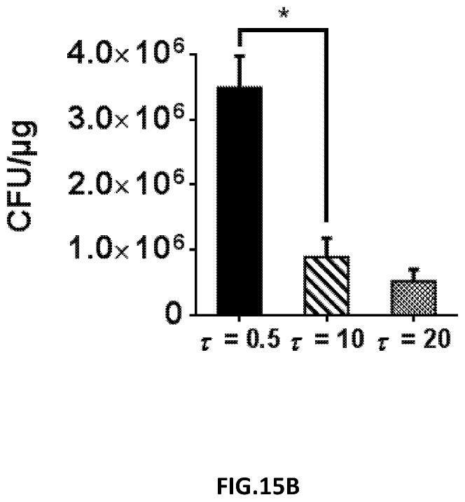

Next, the TEC/DMF setup can be modeled using COMSOL and temperature curves can be simulated for heat shock protocols on device. The efficacy of the COMSOL simulation may be validated by tests involving two temperature probes placed within the heat block of the device setup as well as between the ITO and surface of the DMF chip placed above the temperature block (ie. where a droplet would be) (see FIGS. 13C and 13D). This can establish the accuracy and potential offset correction required to reach temperatures at the droplet position of the TEC/DMF setup. Using a script written in python, the setpoint of the TEC PID may be programed to follow the transfer function of the tube heat shock conditions with a maximum target of 42° C. Heat shocks may be performed with and without applying a corrective time constant on a DMF device containing a 500 nL Cell/DNA sample and results were compared (see FIGS. 14A and 14B). Additionally, other time constant parameters can be tested for heat shocks on DMF conducted with a maximum target of 37° C. with improved efficiencies (see FIGS. 15A and 15B).

The heat shock protocol tested using 42° C. for 30 seconds is a widely used standard. It is interesting to note that through our observations, samples tend to not reach 42° C. in the 30 second time frame (see 12). This may be due to the heat transfer through the polypropylene microcentifuge tube and fluid of the sample. The resulting time constant is relatively high. By scaling sample sizes down to the micro scale, the time constant will drop and the result will be an increase in maximum temperature experienced by the sample which leads to a decrease in efficiency. Using the PID/TEC setup described, a time constant which is much lower than that of a conventional tube heat shock may be attained. For this, a time constant correction was applied and the results were compared. Initially our aim was to more accurately provide temperatures to a sample on DMF which would resemble heat shock in a tube. Building of this new level of temperature control, different time constants with a maximum setpoint of 37° C. were tested. If lower time constants can be achieved, a lower maximum setpoint may be desirable.

While the applicant's teachings described herein are in conjunction with various embodiments for illustrative purposes, it is not intended that the applicant's teachings be limited to such embodiments as the embodiments described herein are intended to be examples. On the contrary, the applicant's teachings described and illustrated herein encompass various alternatives, modifications, and equivalents, without departing from the embodiments described herein, the general scope of which is defined in the appended claims.

### REFERENCES

1. Laksanasopin, T.; Guo, T. W.; Nayak, S.; Sridhara, A. A.; Xie, S.; Olowookere, 0. O.; Cadinu, P.; Meng, F.; Chee, N. H.; Kim, J., A smartphone dongle for diagnosis of infectious diseases at the point of care. Sci. Transl. Med. 2015, 7 (273), 273rel-273rel.
2. Moore, T. A.; Brodersen, P.; Young, E. W., Multiple Myeloma Cell Drug Responses Differ in Thermoplastic vs PDMS Microfluidic Devices. Anal. Chem. 2017, 89 (21), 11391-11398.
3. Kwon, S.; Cho, C. H.; Kwon, Y.; Lee, E. S.; Park, J. K., A Microfluidic Immunostaining System Enables Quality Assured and Standardized Immunohistochemical Biomarker Analysis. Sci. Rep. 2017, 7, 45968.
4. Choi, K.; Ng, A. H.; Fobel, R.; Chang-Yen, D. A.; Yarnell, L. E.; Pearson, E. L.; Oleksak, C. M.; Fischer, A. T.; Luoma, R. P.; Robinson, J. M., Automated digital microfluidic platform for magnetic-particle-based immunoassays with optimization by design of experiments. Anal. Chem. 2013, 85 (20), 9638-9646.
5. Sinha, H.; Quach, A. B.; Vo, P. Q.; Shih, S. C., An automated microfluidic gene-editing platform for deciphering cancer genes. Lab Chip 2018, 18 (15), 2300-2312.
6. Ng, A. H.; Chamberlain, M. D.; Situ, H.; Lee, V.; Wheeler, A. R., Digital microfluidic immunocytochemistry in single cells. Nat. Commun. 2015, 6, 7513.
7. Shih, S. C.; Goyal, G.; Kim, P. W.; Koutsoubelis, N.; Keasling, J. D.; Adams, P. D.; Hillson, N. J.; Singh, A. K., A versatile microfluidic device for automating synthetic biology. ACS Synth. Biol. 2015, 4 (10), 1151-1164.
8. Gach, P. C.; Shih, S. C.; Sustarich, J.; Keasling, J. D.; Hillson, N. J.; Adams, P. D.; Singh, A. K., A droplet microfluidic platform for automating genetic engineering. ACS Synth. Biol. 2016, 5 (5), 426-433.
9. Liu, R. H.; Stremler, M. A.; Sharp, K. V.; Olsen, M. G.; Santiago, J. G.; Adrian, R. J.; Aref, H.; Beebe, D. J., Passive mixing in a three-dimensional serpentine microchannel. J. Microelectromech. Syst. 2000, 9 (2), 190-197.
10. Oh, K. W.; Park, C.; Namkoong, K.; Kim, J.; Ock, K. S.; Kim, S.; Kim, Y. A.; Cho, Y. K.; Ko, C., World-to-chip microfluidic interface with built-in valves for multichamber chip-based PCR assays. Lab Chip 2005, 5 (8), 845-50.
11. Maeki, M.; Yamazaki, S.; Pawate, A. S.; Ishida, A.; Tani, H.; Yamashita, K.; Sugishima, M.; Watanabe, K.; Tokeshi, M.; Kenis, P. J. A.; Miyazaki, M., A microfluidic-based protein crystallization method in 10 micrometer-sized crystallization space. CrystEngComm 2016, 18 (40), 7722-7727.
12. Froger, A.; Hall, J. E., Transformation of plasmid DNA into E. coli using the heat shock method. J. Visualized Exp. 2007, (6).
13. Shih, S. C.; Gach, P. C.; Sustarich, J.; Simmons, B. A.; Adams, P. D.; Singh, S.; Singh, A. K., A droplet-to-digital (D2D) microfluidic device for single cell assays. Lab Chip 2015, 15 (1), 225-36.
14. Yang, H.; Luk, V. N.; Abelgawad, M.; Barbulovic-Nad, I.; Wheeler, A. R., A world-to-chip interface for digital microfluidics. Anal. Chem. 2008, 81 (3), 1061-1067.
15. Au, A. K.; Bhattacharjee, N.; Horowitz, L. F.; Chang, T. C.; Folch, A., 3D-printed microfluidic automation. Lab Chip 2015, 15 (8), 1934-41.
16. Waheed, S.; Cabot, J. M.; Macdonald, N. P.; Lewis, T.; Guijt, R. M.; Paull, B.; Breadmore, M. C., 3D printed microfluidic devices: enablers and barriers. Lab Chip 2016, 16 (11), 1993-2013.
17. Patrick, W. G.; Nielsen, A. A.; Keating, S. J.; Levy, T. J.; Wang, C.-W.; Rivera, J. J.; Mondragón-Palomino, O.; Carr, P. A.; Voigt, C. A.; Oxman, N., DNA assembly in 3D printed fluidics. PloS one 2015, 10 (12), e0143636.
18. Gong, H.; Woolley, A. T.; Nordin, G. P., High density 3D printed microfluidic valves, pumps, and multiplexers. Lab Chip 2016, 16 (13), 2450-8.
19. Husser, M. C.; Vo, P. Q. N.; Sinha, H.; Ahmadi, F.; Shih, S. C. C., An Automated Induction Microfluidics System for Synthetic Biology. ACS Synth. Biol. 2018, 7 (3), 933-944.
20. Vo, P. Q.; Husser, M. C.; Ahmadi, F.; Sinha, H.; Shih, S. C., Image-based feedback and analysis system for digital microfluidics. Lab Chip 2017, 17 (20), 3437-3446.
21. Tsai, H.-L.; Lin, J.-M., Model Building and Simulation of Thermoelectric Module Using Matlab/Simulink. J. Electron. Mater. 2009, 39 (9), 2105-2111.
22. Shah, G. J.; Ding, H.; Sadeghi, S.; Chen, S.; Kim, C. J.; van Dam, R. M., On-demand droplet loading for automated organic chemistry on digital microfluidics. Lab Chip 2013, 13 (14), 2785-95.
23. Au, S. H.; Shih, S. C.; Wheeler, A. R., Integrated microbioreactor for culture and analysis of bacteria, algae and yeast. Biomed. Microdevices 2011, 13 (1), 41-50.
24. Jebrail, M. J.; Wheeler, A. R., Let's get digital: digitizing chemical biology with microfluidics. Curr. Opin. Chem. Biol. 2010, 14 (5), 574-81.
25. Elvira, K. S.; Leatherbarrow, R.; Edel, J.; deMello, A., Droplet dispensing in digital microfluidic devices: Assessment of long-term reproducibility. Biomicrofluidics 2012, 6 (2), 022003.
26. Gong, J., All-electronic droplet generation on-chip with real-time feedback control for EWOD digital microfluidics. Lab Chip 2008, 8 (6), 898-906.
27. Boles, D. J.; Benton, J. L.; Siew, G. J.; Levy, M. H.; Thwar, P. K.; Sandahl, M. A.; Rouse, J. L.; Perkins, L. C.; Sudarsan, A. P.; Jalili, R., Droplet-based pyrosequencing using digital microfluidics. Anal. Chem. 2011, 83 (22), 8439-8447.
28. Jebrail, M. J.; Sinha, A.; Vellucci, S.; Renzi, R. F.; Ambriz, C.; Gondhalekar, C.; Schoeniger, J. S.; Patel, K. D.; Branda, S. S., World-to-digital-microfluidic interface enabling extraction and purification of RNA from human whole blood. Anal. Chem. 2014, 86 (8), 3856-3862.
29. Kim, H.; Jebrail, M. J.; Sinha, A.; Bent, Z. W.; Solberg, O. D.; Williams, K. P.; Langevin, S. A.; Renzi, R. F.; Van De Vreugde, J. L.; Meagher, R. J., A microfluidic DNA library preparation platform for next-generation sequencing. PLoS One 2013, 8 (7), e68988.
30. Shilton, R. J.; Mattoli, V.; Travagliati, M.; Agostini, M.; Desii, A.; Beltram, F.; Cecchini, M., Rapid and controllable digital microfluidic heating by surface acoustic waves. Adv. Funct. Mater. 2015, 25 (37), 5895-5901.
31. Prakash, R.; Pabbaraju, K.; Wong, S.; Wong, A.; Tellier, R.; Kaler, K. V., Multiplex, quantitative, reverse transcription per detection of influenza viruses using droplet microfluidic technology. Micromachines 2014, 6 (1), 63-79.
32. Casquillas, G. V.; Fu, C.; Le Berre, M.; Cramer, J.; Meance, S.; Plecis, A.; Baigl, D.; Greffet, J.-J.; Chen, Y.; Piel, M., Fast microfluidic temperature control for high resolution live cell imaging. Lab chip 2011, 11 (3), 484-489.
33. Moore, J.; Nemat-Gorgani, M.; Madison, A.; Sandahl, M.; Punnamaraju, S.; Eckhardt, A.; Pollack, M.; Vigneault, F.; Church, G.; Fair, R., Automated electrotransformation of Escherichia coli on a digital microfluidic platform using bioactivated magnetic beads. Biomicrofluidics 2017, 11 (1), 014110.
34. Madison, A. C.; Royal, M. W.; Vigneault, F.; Chen, L.; Griffin, P. B.; Horowitz, M.; Church, G. M.; Fair, R. B., Scalable Device for Automated Microbial Electroporation in a Digital Microfluidic Platform. ACS Synth. Biol. 2017, 6 (9), 1701-1709.
35. Campbell, A.; Bauchart, P.; Gold, N. D.; Zhu, Y.; De Luca, V.; Martin, V. J., Engineering of a nepetalactol-producing platform strain of Saccharomyces cerevisiae for the production of plant seco-iridoids. ACS Synth. Biol. 2016, 5 (5), 405-414.
36. Potvin-Trottier, L.; Lord, N. D.; Vinnicombe, G.; Paulsson, J., Synchronous long-term oscillations in a synthetic gene circuit. Nature 2016, 538 (7626), 514.
37. Khanna, M.; Stotzky, G., Transformation of Bacillus subtilis by DNA bound on montmorillonite and effect of DNase on the transforming ability of bound DNA. Appl. Environ. Microbiol. 1992, 58 (6), 1930-1939.
38. Lacks, S. A.; Ayalew, S.; De La Campa, A. G.; Greenberg, B., Regulation of competence for genetic transformation in Streptococcus pneumoniae: expression of dpnA, a late competence gene encoding a DNA methyltransferase of the DpnII restriction system. Mol. Microbiol. 2000, 35 (5), 1089-1098.
39. Rahimzadeh, M.; Sadeghizadeh, M.; Najafi, F.; Arab, S.; Mobasheri, H., Impact of heat shock step on bacterial transformation efficiency. Mol. Biol. Res. Commun. 2016, 5 (4), 257.
40. Moore, M. E.; Lam, A.; Bhatnagar, S.; Solnick, J. V., Environmental determinants of transformation efficiency in Helicobacter pylori. J. Bacteriol. 2014, 196 (2), 337-344.
41. Asif, A.; Mohsin, H.; Tanvir, R.; Rehman, Y., Revisiting the mechanisms involved in calcium chloride induced bacterial transformation. Front. Microbiol. 2017, 8, 2169.
42. Brooke, R.; Hopkins, J.; Laflamme, E.; Wark, P., Effects of temperature-induced changes in membrane composition on transformation efficiency in E. coli DH5α. J. Exp. Microbiol. Immunol. 2009, 13, 71-74.
43. Wang, M.; Liu, Y.; Zhang, C.; Liu, J.; Liu, X.; Wang, L.; Wang, W.; Chen, H.; Wei, C.; Ye, X., Gene editing by co-transformation of TALEN and chimeric RNA/DNA oligonucleotides on the rice OsEPSPS gene and the inheritance of mutations. PLoS One 2015, 10 (4), e0122755.
44. Ghosh, C.; Gupta, R.; Mukherjee, K. J., An inverse metabolic engineering approach for the design of an improved host platform for over-expression of recombinant proteins in Escherichia coli. Microb. Cell Fact. 2012, 11 (1), 93.
45. Miller, E. M.; Wheeler, A. R., A digital microfluidic approach to homogeneous enzyme assays. Anal. Chem. 2008, 80 (5), 1614-1619.
46. Chen, Q.; Groote, R.; Schonherr, H.; Vancso, G. J., Probing single enzyme kinetics in real-time. Chem. Soc. Rev. 2009, 38 (9), 2671-2683.
47. Ciulli, A.; Abell, C., Fragment-based approaches to enzyme inhibition. Curr. Opin. Chem. Biol. 2007, 18 (6), 489-496.
48. Ven, K.; Safdar, S.; Dillen, A.; Lammertyn, J.; Spasic, D., Re-engineering 10-23 core DNA- and MNAzymes for applications at standard room temperature. Anal. Bioanal. Chem. 2018, 1-11.
49. Srinivasan, V.; Pamula, V. K.; Fair, R. B., An integrated digital microfluidic lab-on-a-chip for clinical diagnostics on human physiological fluids. Lab Chip 2004, 4 (4), 310-315.
50. Choi, K.; Mudrik, J. M.; Wheeler, A. R., A guiding light: spectroscopy on digital microfluidic devices using in-plane optical fibre waveguides. Anal. Bioanal. Chem. 2015, 407 (24), 7467-7475.
51. Luk, V. N.; Wheeler, A. R., A digital microfluidic approach to proteomic sample processing. Anal. Chem. 2009, 81 (11), 4524-4530.
52. Srinivasan, V.; Pamula, V. K.; Fair, R. B., Droplet-based microfluidic lab-on-a-chip for glucose detection. Anal. Chim. Acta 2004, 507 (1), 145-150.
53. Sista, R. S.; Eckhardt, A. E.; Wang, T.; Graham, C.; Rouse, J. L.; Norton, S. M.; Srinivasan, V.; Pollack, M. G.; Tolun, A. A.; Bali, D., Digital microfluidic platform for multiplexing enzyme assays: implications for lysosomal storage disease screening in newborns. Clin. Chem. 2011, 57(10), 1444-51.
54. Peralta-Yahya, P. P.; Keasling, J. D., Advanced biofuel production in microbes. Biotechnol. J. 2010, 5 (2), 147-162.
55. Gladden, J. M.; Park, J. I.; Bergmann, J.; Reyes-Ortiz, V.; D'haeseleer, P.; Quirino, B. F.; Sale, K. L.; Simmons, B. A.; Singer, S. W., Discovery and characterization of ionic liquid-tolerant thermophilic cellulases from a switchgrass-adapted microbial community. Biotechnol. Biofuels 2014, 7 (1), 15.
56. Dave, B. R.; Sudhir, A. P.; Subramanian, R., Purification and properties of an endoglucanase from Thermoascus aurantiacus. Biotechnol. Rep. 2015, 6, 85-90.
57. Parry, N.; Beever, D.; Owen, E.; Nerinckx, W.; Claeyssens, M.; Van Beeumen, J.; Bhat, M., Biochemical characterization and mode of action of a thermostable endoglucanase purified from Thermoascus aurantiacus. Arch. Biochem. Biophys. 2002, 404 (2), 243-253.

What is claimed is:

1. A microfluidic device comprising:
a first plate comprising:
a cell culture region for maintaining a cell culture; and
a reservoir for storing a liquid reagent to induce at least a portion of the cell culture or to mix with other reagents;
a second plate spaced apart from the first plate, the second plate defining one or more openings extending through the second plate; and
a reagent well coupled to the second plate, the reagent well configured to refill the reservoir on the first plate with the liquid reagent via the one or more openings of the second plate.

2. The microfluidic device of claim 1, wherein the reagent well is vertically spaced apart from the reservoir.

3. The microfluidic device of claim 1 or claim 2, wherein the reagent well is laterally spaced apart from the reservoir.

4. The microfluidic device of any one of claims 1 to 3, wherein the reagent well is coupled to a top of the second plate.

5. The microfluidic device of any one of claims 1 to 4, wherein the reagent well is coupled to a top of the second plate directly above the one or more openings of the second plate.

6. The microfluidic device of any one of claims 1 to 5, wherein the first plate comprises an ancillary reservoir and the reagent well is configured to deliver the liquid reagent to the ancillary reservoir.

7. The microfluidic device of claim 6, wherein the reagent well is aligned with a center or an edge of the ancillary reservoir.

8. The microfluidic device of any one of claims 1 to 7, wherein the reagent well comprises a plunger for dispensing droplets of the liquid reagent.

9. The microfluidic device of claim 8, wherein the plunger is a screw configured to fit within the reagent well and the liquid reagent is dispensed from the reagent well by applying an external pressure to the plunger.

10. The microfluidic device of claim 9, wherein the external pressure is a downward pressure.

11. The microfluidic device of any one of claims 8 to 10, wherein the reagent well is 3D printed and the plunger is fitted within the reagent well.

12. A microfluidic system comprising:
a microfluidic device comprising:
a first plate comprising:
a cell culture region for maintaining a cell culture; and
a reservoir for storing reagents to induce at least a portion of the cell culture or to mix with other reagents; and
a second plate spaced apart from the first plate, the second plate defining one or more openings extending through the second plate; and
a thermoelectric module (TEM) coupled to the first plate or the second plate, the TEM for controlling a temperature of the device.

13. The system of claim 12, wherein the TEM is coupled to the second plate.

14. The system of claim 12 or claim 13, wherein the TEM is positioned underneath the second plate.

15. The system of any one of claims 12 to 14, wherein the TEM is a Peltier module.

16. The system of any one of claims 12 to 15, wherein the microfluidic device further comprises a temperature sensor to receive temperature information from the TEM.

17. The system of any one of claims 12 to 16, wherein the TEM is controlled by a closed-loop temperature model.

18. The system of any one of claims 12 to 17, further comprising a PID controller to control the TEM.

19. The system of any one of claims 12 to 18, further comprising an aluminum heat block.

20. The system of claim 19, wherein the aluminum heat block is positioned between the TEM and the microfluidic device.

21. The system of claim 20, wherein the temperature sensor is positioned within the aluminium heat block.

22. A method of controlling a temperature of a region of a microfluidic device, the method comprising:
comparing a set point temperature to a measured temperature, the measured temperature measured by a temperature sensor;
determining a temperature set point error based on the measured temperature and the set point temperature;
providing the temperature set point error to a proportional-integral-derivative (PID) controller; and
calculating, by the PIC controller, an output value based on the temperature set point error and a set of parameters for proportional, integral and derivative temperature control.

23. The method of claim 22 further comprising measuring the measured temperature with a temperature sensor positioned in a heat block before comparing the set point temperature to the measured temperature.

24. The method of claim 23, wherein the set point temperature is a value subtracted from the measured temperature from the temperature sensor.
